(19) United States
(12) Patent Application Publication
(10) Pub. No.: US 2023/0086561 A1
(43) Pub. Date: Mar. 23, 2023

(54) IMPLANTABLE GUIDE ELEMENT AND METHODS OF FABRICATION AND USE THEREOF

(71) Applicant: NATIONAL UNIVERSITY OF SINGAPORE, Singapore (SG)

(72) Inventors: Agata BLASIAK, Singapore (SG); Amitabha LAHIRI, Singapore (SG); Nitish Vyomesh THAKOR, Singapore (SG)

(21) Appl. No.: 17/784,278

(22) PCT Filed: Nov. 30, 2020
(86) PCT No.: PCT/SG2020/050706

§ 371 Date: Jun. 10, 2022

(30) Foreign Application Priority Data
Dec. 10, 2019 (SG) 10201911928W

(51) Int. Cl. A61N 1/05; A61B 5/24

(52) U.S. Cl. CPC A61N 1/05; A61B 5/24

(57) ABSTRACT

An implantable guide element comprises a main body formed from a biocompatible material. One or more grooved surface structures are provided on and/or within the main body, each grooved surface structure comprising one or more grooves for directionally guided growth of fibro-axonal tissue. At least one of the one or more grooved surface structures may form a channel along or within the main body, within which an electrode is disposed in spaced relationship from a wall of the channel along at least part of its length.

### CROSS-REFERENCE TO RELATED APPLICATIONS

This application is a 371 U.S. National Stage of International Application No. PCT/SG2020//050706, filed Nov. 30, 2020, and claims priority to Singapore Patent Application No. 10 2019 11928W, filed Dec. 10, 2019, and the disclosures of which are herein incorporated by reference in their entirety.

### TECHNICAL FIELD

The present disclosure relates to an implantable guide element, and a method of fabricating an implantable guide element. The present disclosure also relates to uses of an implantable guide element, such as for assistance in repair of nerve injury, and as a neural interface element.

### BACKGROUND

Over recent years, neurotechnology has emerged as a path forward toward augmentation of human abilities in both sick and healthy individuals. Neurotechnology is expected to deliver neuro-electronic integration for bionic applications, such as prosthetics for patients with amputations, and exoskeletons for patients with paralysis. For such applications, there is a need to develop an interface to the peripheral nerve such as the sciatic nerve (lower limb), or radial and ulnar nerve (upper limb), to record, stimulate or serve as a bridge or scaffold for a cut or injured nerve. Other developments are internal implants interfaced to visceral nerves (pelvic, pudendal) such as those controlling the urinary bladder for age related incontinence, interface to the vagus for many clinical indications such as epilepsy, or to the phrenic nerve for diaphragm control for respiratory paralysis by recording or stimulating these nerves.

When applied in healthy individuals, neuro-electronic integration can improve abilities of an individual and, for example, support independence and mobility in aging populations via exoskeleton mechanisms or robotic assistants. Neuro-electronic integration requires nerve interfaces to provide a bridge or an interface for electronic recording, or for stimulation via neuroelectronic devices for achieving neuromodulation.

The key to neurotechnology systems is a high-quality integration between biotic and abiotic elements, that is, nerves and the engineered system. Such integration has to be stable, long lasting and well tolerated by the body. Neural interfaces for peripheral nerves face an additional challenge of lack of physical anchorage between the nerve tissue and the implant.

The major hurdle in the development of a neuro-prosthesis has been the biological challenge of creating a stable, long-term bioelectrical interface. Currently, simple strategies rely on an extraneural or intraneural interface with the axons achieved through direct physical contact or penetration respectively. For example, Flat Interface Nerve Electrodes (FINE) are applied to the exterior of the nerve and function through enhancement of surface contact by physically compressing the nerve. The FINE electrodes do not inflict penetrative trauma to the nerves but the signal quality and specificity to capture nerve signals is highly constrained. Microelectrode arrays have needle-like electrodes that directly penetrate a nerve for potentially better quality and more specific signals. These electrodes result in immediate penetrative trauma to the nerve, and have limited lifespan due to progressive decline in conductivity. This decline results from trauma secondary to electrode micro-motion within the relatively soft neural substrate, and progressive insulation by fibrosis around the implant. Longitudinal Intrafascicular Electrodes (LIFE) and Transverse Intrafascicular Multichannel Electrode (TIME) are soft strip electrodes that are inserted within the nerve tissue. These electrodes are easier to insert into a nerve and again may offer specificity, but they also induce trauma and fibrosis within the nerve and are used primarily for nerve stimulation rather than recording. Several biological strategies are under investigation to create stable neural interfaces. Regenerative peripheral nerve interfaces (RPNIs) involve embedding cut ends of the peripheral nerves into muscle grafts to resolve neuroma pain. RPNIs translate neural signals into large amplitude myoelectric activity, which, in effect, produces a many-fold amplification of the neural signal. RPNIs however do not represent a true neural interface and reduce the multitude of axonal signals available in a fascicle to significantly fewer compound muscle action potentials (CMAPs).

Regenerative neural interfaces (RNIs) are a distinct group that incorporate tissue-engineering strategies to create direct interfaces between a nerve and an electrode. These electrodes are designed such that they make contact with the regenerating axons, typically from a peripheral nerve. Various techniques have been developed to enhance axon growth across electrodes. These include the use of material coatings, topographic cues, and incorporation of trophic chemoattractant factors. These interfaces create a functional contact with the axons, but their functional longevity is compromised by the fibrosis initiated by the implant material itself.

Indeed, innervation of a synthetic electrode to establish a stable electrophysiologic contact and the capability to access the signals for control of robotic prosthesis remain unsolved challenges.

A particular unresolved challenge in relation to previous attempts to create neural interfaces is fibrosis. For example, in previously conceived implants, a flat electrode is provided in the wall of a microchannel device for conduction of signals. However, fibrotic growth on the electrode surface creates insulation between the axons and the electrode surface. Various materials have been used to coat the electrodes to enhance axonal guidance, but this does not address the issue of fibrotic sequestration.

It is desirable therefore to address or alleviate at least one of the above challenges, or at least to provide a useful alternative.

### SUMMARY

The present disclosure relates to an implantable guide element, comprising: a main body formed from a biocompatible material; and one or more grooved or ridged surface structures on and/or within the main body, each grooved or ridged surface structure comprising one or more grooves or ridges for directionally guided growth of, and encapsulation by, fibro-axonal tissue.

Advantageously, the guided growth facilitated by the grooves results in a structure having a sheet like configuration of fibro-axonal tissue, making the axons more accessible and organized than was previously possible.

Advantageously, the main grooved/ridged body provides a core for guided encapsulation by fibrous and axonal (neural) composite tissue creating a fibro-axonal/fibro-neural composite having a laminar sheet like configuration, making the axons more accessible and organized than was previously possible.

In certain embodiments, one or more grooves or ridges may have a coating comprising one or more of: charge changing molecules, adhesion molecules, growth factors, and supportive cells. The coating may have a concentration gradient along the one or more grooves.

In certain embodiments, two or more grooves may have different respective coatings suitable for promoting adhesion and/or growth of different respective cell types.

The main body may be an elongate structure having an axis, and the at least one groove or ridge may be aligned generally along the axis.

In certain embodiments, at least one of the one or more grooved surface structures forms a channel along or within the main body. An electrode may be disposed within the channel (or multiple electrodes may be disposed within respective channels), and spaced from a wall of the channel along at least part of its length.

The electrode may have a helical portion, for example. A helical electrode is particularly advantageous as it ensures that the electrode intersects the axonal tissue at multiple points, thus maintaining a stable and consistent electrical connection.

In certain embodiments, the main body has a tapered end for insertion into a nerve.

In certain embodiments, the main body has a rigid base portion. In embodiments that contain one or more electrodes, the rigid base portion may house a connector of the electrode (or connectors of respective electrodes). In any case, the rigid base portion may house one or more of an innervation target, one or more molecular growth factors, a source of a magnetic or electromagnetic field, and one or more guidance molecules.

In certain embodiments, the biocompatible material is VeroClear.

The present disclosure also relates to a method of fabricating a guide element for implantation into a subject, comprising: obtaining dimensional measurements of a nerve of the subject; and forming, in accordance with the dimensional measurements by an additive manufacturing method, using a biocompatible material: a main body; and one or more grooved or ridged surface structures on and/or within the main body, each grooved or ridged surface structure comprising one or more grooves or ridges for directionally guided growth of, and encapsulation by, fibro-axonal tissue. The method may comprise applying a coating to one or more grooves, the coating comprising one or more of: charge changing molecules, adhesion molecules, growth factors, and supportive cells. The coating may be applied with a concentration gradient.

The method may comprise applying different respective coatings suitable for promoting adhesion and/or growth of different respective cell types to two or more grooves.

The method may comprise providing an electrode within the channel and spaced from a wall of the channel along at least part of its length. The electrode may have a helical portion.

The method may comprise forming the main body with a tapered end for insertion into a nerve.

The method may comprise forming the elongate body with a rigid base portion. In some embodiments, the method may comprise housing a connector of the electrode within the rigid body portion. Whether or not an electrode is provided in the guide element, the method may comprise inserting one or more of the following into the rigid base portion: an innervation target, one or more molecular growth factors, a source of a magnetic or electromagnetic field, and one or more guidance molecules.

Also disclosed herein is a method of treating an injured or divided nerve, comprising: providing at least one implantable guide element as disclosed herein; and positioning the at least one fibro-axonal guide element alongside and/or within the injured or divided nerve; whereby fibro-axonal tissue is caused to grow from said injured nerve along grooves of the, or each, implantable guide element.

The method may comprise positioning a first end of the fibro-axonal guide element within a first portion of the injured nerve, and positioning a second end of the neural guide element within a second portion of the injured nerve.

The method may comprise encasing the first and second portions of the injured nerve with a coaptation sleeve.

Further disclosed herein is a method of treating an injured nerve, comprising: providing an implantable guide element as disclosed herein; coating at least one groove of the implantable guide element with a therapeutic agent; and positioning the at least one neural guide element alongside and/or within the injured nerve.

### BRIEF DESCRIPTION OF THE DRAWINGS

Some embodiments of an implantable guide element, and methods of its fabrication and use, in accordance with present teachings will now be described, by way of non-limiting example only, with reference to the accompanying drawings in which:

FIG. 1 is a side view of an implantable guide element according to certain embodiments.

FIG. 2 is a cross-section through the line a of FIG. 1.

FIG. 3 is a cross-section through the line b of FIG. 1.

FIG. 4 is a cross-section through the line c of FIG. 1.

FIG. 5 is a cross-section through the line d of FIG. 1.

FIG. 6 is a side view of the implantable guide element of FIG. 1, showing an electrode positioned in one of the channels thereof.

FIG. 7 is a side view of the electrode.

FIG. 8 is a cross-section through the line 8 of FIG. 6.

FIG. 9 is a schematic view of the implantable guide element being implanted in a nerve.

FIG. 10 is a cross-sectional view of an implantable guide element according to alternative embodiments.

FIG. 11 is a cross-sectional view of an implantable guide element according to yet further embodiments.

FIG. 12 is a schematic view of the implantable guide element of FIG. 10 being implanted at the site of a nerve injury.

FIG. 13 is an alternative schematic view of the implantable guide element of

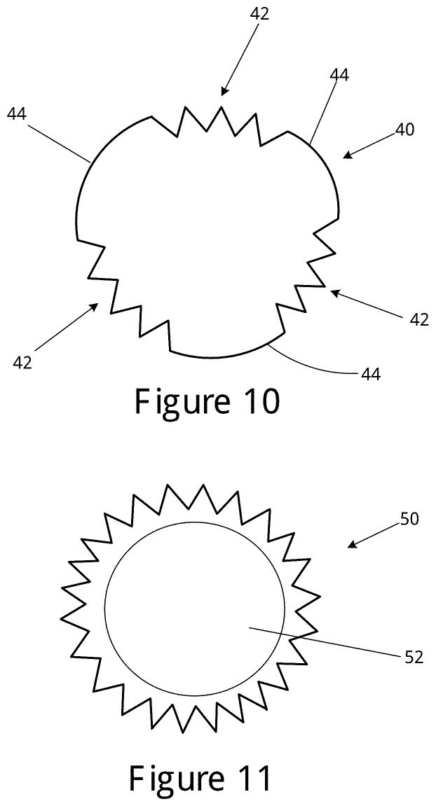

FIG. 10 being implanted at the site of a nerve injury.

FIG. 14(A) is a schematic side view of another embodiment of a guide element.

FIG. 14(B) is a cross-section through the line B of FIG. 14(A).

FIG. 14(C) is a cross-section through the line C of FIG. 14(A).

FIG. 14(D) is a cross-section through the line D of FIG. 14(A).

FIG. 15(A) is a cross-sectional view through another embodiment of a guide element.

FIG. 15(B) is a side view of the guide element of FIG. 15(A) encasing a nerve.

FIG. 15(C) is a cross-sectional view of the guide element of FIG. 15(A) encasing the nerve.

FIG. 16 is an isometric view of another embodiment of a guide element.

FIG. 17 is an isometric view of a further embodiment of a guide element.

FIG. 18 is a cross-sectional view of a further embodiment of a guide element.

FIG. 19 is a cross-sectional view of a yet further embodiment of a guide element.

FIG. 20 is a cross-sectional view of a yet further embodiment of a guide element.

FIG. 21 shows images of neuronal adhesion and axon growth on a textured substrate.

FIG. 22 shows data relating to adhesion and alignment of fibroblasts on a textured substrate in vitro.

FIG. 23 shows data relating to fibro-neuronal growth and axon alignment on a textured substrate in vitro.

FIG. 24 shows a schematic representation of implant placement, with the apex placed in the interfascicular space of the nerve (n) with the coils and columns outside the nerve.

FIG. 25 shows a schematic representation of an electrophysiologic set-up four months after implant. Stimulus (st) applied to the cortex, generated a compound action potential (CAP) carried by the nerve (n) which passed through the interface (n). The interface was connected to an amplifier (A′) and a recorder (R).

FIG. 26 shows: A,B—Sample neural recordings across the interface in channels 1&2 generated following cortical stimulus amplitudes of 260 μA. Complete range of signals is shown in FIGS. 27 and 28 (Y axis: Amplitude in 10-5 μV, X axis: time in seconds). C—Average CAP peak voltage and triggering probability. Two subplots represent two channels that were used in the recording. Bars indicate the average CAP peak voltage of the signals that exceed 10 μV. Diamond markers indicate the probability of the signals that exceed 10 μV over the total number of stimulation trains in each amperage (Y-axis: average CAP peak voltage in μV and X-axis: stimulation amplitude in μA). D—SNR of 2 channels with increasing stimulation current. SNR of evoked signals observed in both channels are in scale of stimulation amplitudes, where the maximum SNR are 11.03 and 12.06 dB in channels 1 and 2 respectively (Y-axis: SNR in dB, and X-axis: stimulation amplitude in μA). E—Single stimulus series to show the stimulus train and the intervening action potentials in two channels. Y axis: signal amplitude in 10-5 Volts, X-axis time in 0.05 seconds. A circle denotes the beginning of the stimulus train and an X denotes the termination.

FIG. 27 shows gross morphology of the nerve-implant interface 4 months following implantation. A: Implant removed en-bloc with silicone cylinder (sic) and the adjacent segment of the ulnar nerve (n). B: Morphology with silicone cylinder removed. The nerve (n) seen firmly connected to the implant (vc). B1: Magnified view of nerve-implant interface against 1 mm grid: tissue growth (fxg) from the nerve seen encapsulating the implant body and growing into the channels. B2: Magnified view of tissue growth within the implant channel demonstrating encasement of the platinum coil microelectrode within the tissue. C: Extraction of the implant from the tissue: Implant (vc), nerve (n) New tissue growth (fxg) from the nerve over the implant. D: Zones for histological examination. Normal nerve (n), transition zone (t) between normal nerve and fibro-axonal (fxg) tissue growth. Proximal segment of the tissue growth (fxp), distal tip of tissue growth (fxd).

FIG. 28 shows histology of normal nerve and fibro-axonal growth. A: Reference specimen divided into proximal fibroaxonal growth (fxp), transition zone (t), normal nerve (n) for sectioning (solid arrows indicating the direction of growth). A1 (Fibro axonal growth): H&E stain: reconfiguration of axons and fibrous tissue to the cross-sectional shape of the implant (inset) arrow showing growth within the channel. A2: Corresponding section in Neurofilament (NF) labeling showing axonal clusters Ax (dark brown) in a laminar arrangement, forming the intermediate layer and following the contour of the implant. Layers of fibrous tissue (Fi) demonstrated by purple background stain seen sandwiching the axonal layer. B1&B2 (Transition zone): H&E and NF labelled specimens respectively, at transition zone between the nerve and fibro-axonal growth on the body of the implant. The holes correspond to the tips of the columns of the implant. C1 &C2: Normal nerve H&E and NF labelled sections of the normal nerve demonstrating the normal fascicular arrangement of axons (Ax). The stain shows fine points indicating individual axons without clustering. The epineurium (Epi) is seen as lose connective tissue in contrast to the dense fibrous layer in the fibroaxonal growth.

FIG. 29 shows sections from the distal tip pf fibro-axonal growth (fxd) with Neurofilament (NF) antibody labelling: A: intact specimen for reference. Al: NF labelled low power transverse section through the tip showing sheet like tissue following the contour of the implant and axonal layer (dark brown stain) within the layers of fibroblasts (purple stain). A2: same section in high magnification showing dark brown axonal clusters (ax) in laminar arrangement within layers of fibroblasts tissue (purple nuclei) following the contour of the implant. B1: NF stained low power longitudinal section of the tip showing fibro-axonal growth in two opposite channels and axonal layer ax(dark brown stain). B2: High power image of the tip of the growth in longitudinal section, showing the extent of the axonal layer(ax) sandwiched between fibroblasts layer (purple stain). C1: control specimen without the implant showing a typical neuroma formation from the ulnar nerve (n). C2: NF labelled transverse section demonstrating random arrangement of axonal clusters (arrows) within a background of fibrous tissue (purple). D: NF labeled section of normal nerve showing bundles of axons (dark brown stain,) without any fibroblasts within the nerve and surrounding layer of loose connective tissue epineurium (purple stain).

FIG. 30 shows fibroaxonal growth in longitudinal section: A: Fibro axonal growth over the implant. A polypropylene suture (blue) is inserted to demonstrate the hollow space within the growth which was occupied by the implant. A1&A2: reconstructed histology of the entire specimen demonstrating in NF and S100 antibody labelling respectively. The specimen shows tissue growth within two opposite channels of the implant and co-location of axons (NF, A1) and Schwann cells (S-100, A2). The central hollow represents the body of the implant. B1: Typical solid neuroma formed at the end of the ulnar nerve in the control group without the implant. B2: Longitudinal section of the neuroma showing clusters of dark brown stained axons (arrows) encased within fibrous tissue. And the absence of parallel arrangement of axons. C: Normal nerve labeled with NF stain showing parallel bundles of axons with a thin layer of epineurium.

FIG. 31 shows a conceptual illustration of fibroaxonal tissue and its relationship to electrodes. A: placement of the spire of the implant within the inter-fascicular space of the nerve (n), with the body and coil electrodes(pt) outside the nerve. B: Fibro-axonal growth (fxg) into the channels containing the coiled electrodes. C: cross section showing channels and the location of electrodes (green circles). D: showing encapsulation of the implant (vc) and electrodes (El), (green circles) within fibroaxonal growth containing fibrous layer (Fi orange) and axonal layer (Ax, dotted lines). The coil electrode within the channels intersects with the axonal layer at multiple points and conducts action potential. The relative diameter of the coil allows the coil to cross the entire thickness of the fibroaxonal tissue. E: cross section of a hypothetical channel device where the electrode (El, green circle) is incorporated within the surface of the channel (green). Fibrosis (Orange) on the wall around the axons will result in insulation of the axons from the conductive surface. F: Color enhanced histology to demonstrate the fibrous layer (Fi) in purple and axons (brown stain) sandwiched between layers of fibrous tissue. Conventional placement of electrodes (green semicircles) on the Internal (El1) or external (El2) surface as in channel devices results in separation from axonal layer by the fibrous tissue deposition, while the coil El (green full circle) represents intersects the axonal layer (Ax) within the fibrous tissue at multiple points and makes contact with the axonal layer in spite of fibrosis surrounding the axons.

FIG. 32 shows a long-term impedance study for an electrode. The electrode construct was soaked in PBS at 67° C. for over 15 weeks. The initial decrease of the impedance is attributed to metal-fluid interface equilibration. The impedance remained stable for over 15 weeks.

FIG. 33 shows averaged signals from Channels 1&2. Overlay of averaged signals from multiple trials at cortical stimulus currents from 100 μA to 260 μA. (Y axis: markings at 2×10-5 V, X axis markings at 0.01 seconds). Consistent recruitment is seen from stimuli of 180 μA. All signal amplitudes are input referred.

FIG. 34 shows CD45 antibody labelling for neutrophils: Negative staining for CD45. Rare neutrophils (dark stain, arrows) are seen in the histologic section indicating absence of ongoing inflammatory response around the implant. Background light stain represents fibroblast nuclei.

### DETAILED DESCRIPTION

Embodiments of an implantable guide element (for example, usable as a neural guide element) comprise an elongate body having surface texturing comprising a plurality of grooves that extend along at least part of the elongate body. Advantageously, it has been found that providing such grooves or ridges encourages fibroblast growth and adhesion on the guide surface, and subsequent axon growth in directed fashion, thus enabling faster healing when the guide element is used for treatment of nerve injury or division, or faster or reliable attachment to an electrode of the guide element when used as part of a neural interface. Additionally, the grooves of the guide element enable a defined and stable position of a nerve with which the guide element is used, e.g., in contact with an electrode, without causing damage to the nerve. In particular, the grooves of the guide element provide guidance to nerve fibres, including axons, providing increased surface area for adhesion and a conduit or core for directionality.

Further, by providing structures that encourage fibroblast adhesion, it becomes possible to attach a guide at a specific site on the nerve without requiring any additional anchoring or attachment means.

The guide element of the present disclosure induces laminar organization of fibro-axonal tissue over its surface, providing a stable and consistent physical support and axonal guidance.

As used herein, the term “fibro-axonal tissue” refers to a composite of axons and fibrous tissue, wherein the axons are trapped within a mass of the fibrous tissue, but still retain their electrophysiological activity.

In addition to the structural growth/adhesion promotion provided by the grooves of the guide element, further enhancement of growth and/or adhesion may be achieved by providing a coating on the surface of one or more of the grooves. For example, the coating may comprise one or more of: charge changing molecules, adhesion molecules, growth factors, and supportive cells. Such coatings may be applied with a concentration gradient to stimulate growth (for example) in a particular direction, for example.

The implantable guide element is formed from a biocompatible material that supports cellular adhesion. In some embodiments, the implantable guide element may be formed from a material that is both transparent and biocompatible. An example of such a material is acrylic, including various acrylic formulations. The material may include one or more (meth)acrylic compounds and acrylate based polymers, such as one or more (meth)acrylate monomers, oligomers, and polymers and other acryl based formulations, for example a combination including: isobornyl acrylate, acrylic monomer, urethane acrylate, epoxy acrylate, acrylate oligomer.

For example, an acrylic formulation under the product name: VeroClear™ RGD810 of Stratasys Limited may advantageously be used to form the neural guide element by additive manufacturing. Fabrication by additive manufacturing, also commonly known as 3D printing, enables rapid production of customised implantable guides with size and shape, along with its inner and outer texture according to the specific requirements of the subject in which the guide will be implanted, and a specific function the guide is expected to have. For example, the guide element may be fabricated in accordance with a specific nerve type of the subject, or the specific dimensions of the nerve to which the guide is to be attached or inserted.

It has surprisingly been found that VeroClear, which is a commonly used 3D-printing material, has exceptional biocompatibility that allows cell adhesion and organisation into a compact layer, and does not induce significant inflammation. VeroClear also has physical properties that make it particularly advantageous for use as a guide element, such as being sufficiently rigid and robust to guide fibro-neuronal outgrowth and to house delicate electronic components upon implantation into the body, and the added features of transparency and limited autofluorescence that facilitate follow-up that requires continuous monitoring of the health of the nervous tissue and cellular microenvironment in vivo, and to provide compatibility with visual assays ex vivo (upon explantation), such as microscopy. It will be appreciated that other acrylic-based and non-acrylic based materials may also provide similar rigidity, robustness and transparency.

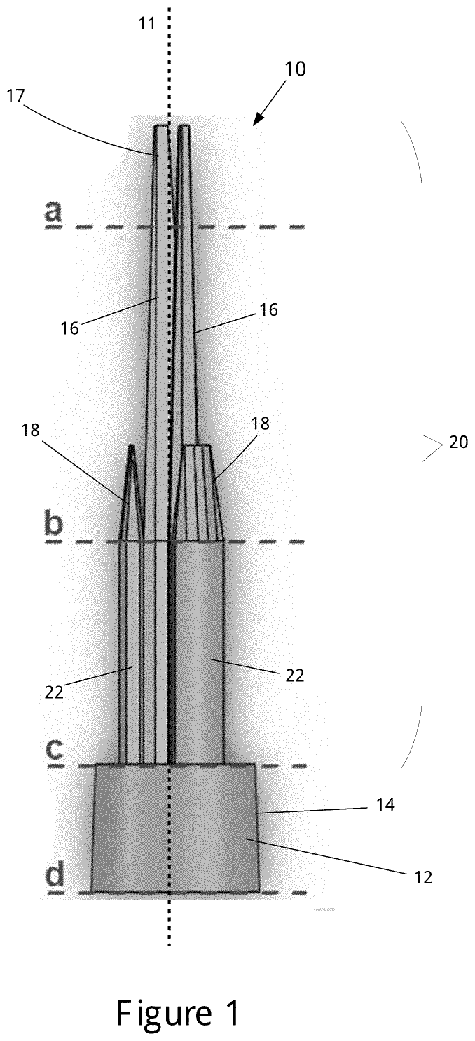

A first embodiment of an implantable guide element will now be described with reference to FIGS. 1 to 8.

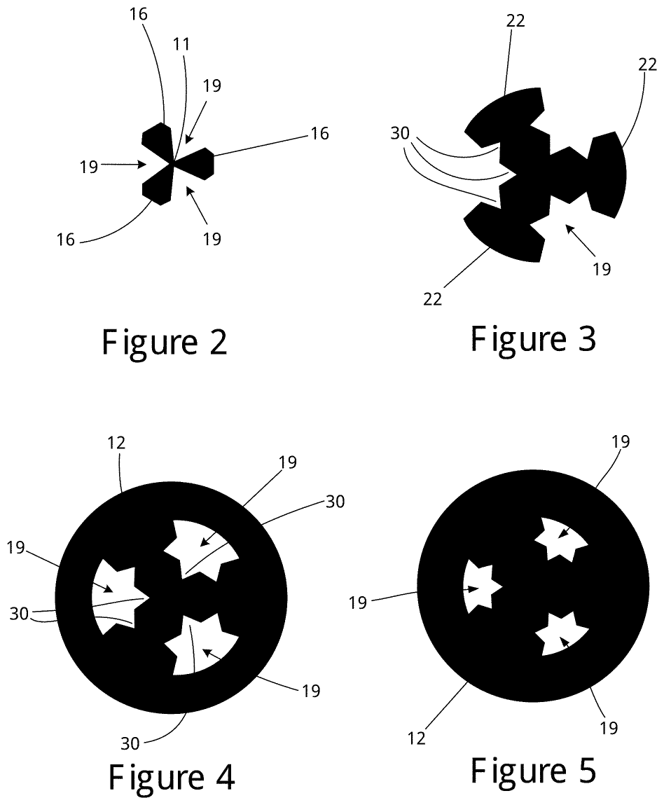

In FIG. 1, an implantable guide element 10 comprises a main body comprising a rigid base portion 12 from which extends a spire 20. The spire 20 comprises a plurality of columns 22 extending from the base 12, and each column 22 has a tapered crown portion 18. Spire 20 also comprises a plurality of needle structures 16 (as seen in the cross-sectional view of FIG. 2) extending from the base 12, each needle structure 16 ending in a tapered end 17. The needle structures 16 meet at a central axis 11 of the guide element 10. It will be appreciated that in some embodiments, a single needle structure 16 may extend from base 12.

Advantageously, the inwardly tapered crown portions 18 of columns 22 assist in guiding the columns 22 into a silicone tube (or tube of like material) that may then be used to attach the guide element 10 to a nerve. To this end, the columns 22 may have a slightly larger outer diameter than the inner diameter of the tube, to ensure a tight friction fit with the tube.

The external surface or surfaces of the needle structure or structures 16 and columns 22 define a plurality of grooved surface structures, as shown in the cross-sectional views in FIGS. 2 to 5. Each grooved surface structure comprises a plurality of grooves 30, and each groove 30 is aligned generally along the central axis 11 of the main body. The grooves 30 provide a substrate for growth of axons along the at least one grooved surface structure.

In particular, as will be shown later with reference to in vivo experimental data obtained by the present inventors, the elongate grooves 30 encourage growth of fibro-neuronal tissue along the length of the elongate body 20 of the fibro-axonal guide element 10. Subsequently, due to secretion by fibroblasts of extra-cellular matrix proteins such as type 1 collagen, structural handles are created for elongating axons, which are also thereby encouraged to grow along the elongate grooves 30.

The grooves 30, or ridges, may have a depth, or height, in the range from about 100 microns to about 1 mm to accommodate nerves; and more particularly, in the range from about 200 microns to 500 microns to accommodate nerve fascicles and nerve fibres. In some embodiments, the grooves 30 may have a depth of about 315 microns. Hereinafter, reference to grooves and ridges will be understood to be interchangeable, with ridges bounding grooves and grooves bounding ridges, unless context dictates otherwise.

In the embodiment of FIGS. 1 to 8, a plurality of channels 19 is formed along the neural guide element 10, each channel containing a grooved structure comprising a plurality of grooves 30. The channels 19 are isolated from each other by virtue of needle structures 16 and columns 22. Accordingly, if the guide element 10 is used as a neural interface, electrodes can be provided within channels 19 (the electrodes being mechanically shielded by columns 22), and separate signals can be recorded and/or transmitted along separate channels 19 once fibro-axonal growth is complete and the nerve to which the guide element is interfaced is thereby in electrical communication with one or more electrodes of the guide element 10. In some embodiments, more or fewer channels 19 than depicted in FIGS. 1 to 8 may be formed; for example, a single channel 19 may be formed in the guide element 10.

The portions of channels 19 that extend through the base 12 may be tapered in a direction extending towards the bottom of the base 12, away from the tip 17. This may assist in stabilising the position of a wire crimp of an electrode inside the base 12, as the channel 19 may be made wide enough at the top of base 12 to insert the wire crimp, and then due to the narrowing of the channel 19 towards the bottom of the base 12, the wire crimp may form a friction fit with the walls of channel 19 in an intermediate position within the base 12.

The neural guide element 10 is formed from a rigid, and biocompatible material, which may be transparent, such as VeroClear as mentioned above. Advantageously, the base portion 12 may thereby act as a protective housing for electronic components that are connected to any electrodes of the guide element, and for delicate connections (such as crimp connectors) between the electrodes and wiring that is used to connect the electrodes to components external to the neural guide element 10 that can then communicate signals external to the body.

Whether or not the guide element contains any electrodes, the rigid base portion 12 may be used to house various electrical, cellular or molecular components for further stimulating nerve tissue growing within the channels 19. For example, the rigid base portion 12 may house an innervation target (such as muscle tissue); one or more molecular growth factors; one or more guidance molecules; and/or a source of a magnetic or electromagnetic field.

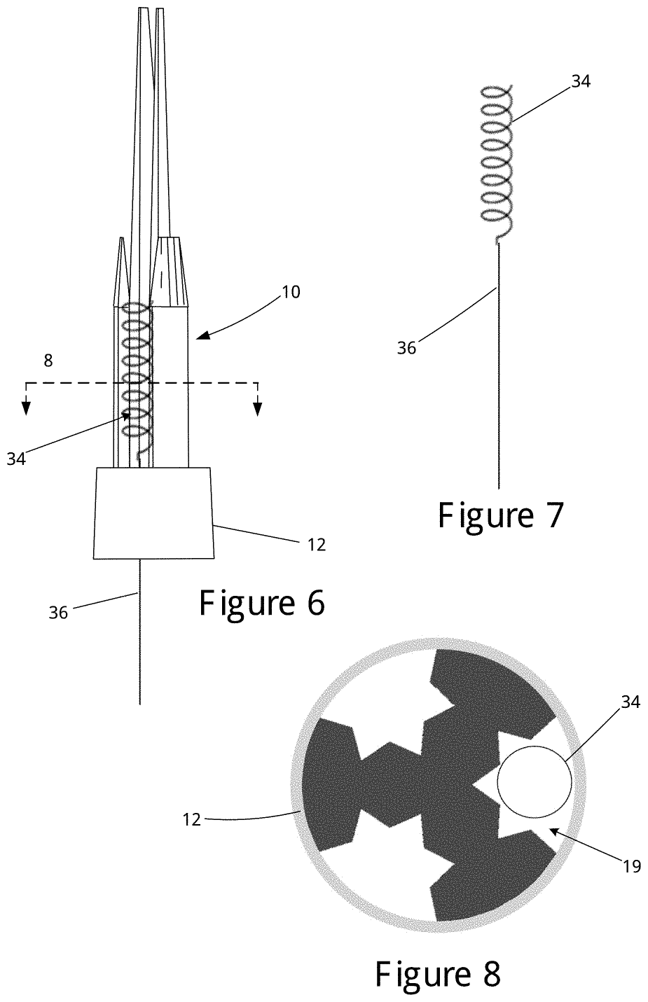

The use of the guide element 10 as a neural interface is depicted in FIGS. 6 to 9. In FIGS. 6 and 8, an electrode 34 is shown positioned within one of the channels 19 of the guide element 10. Electrode 34 may comprise at least a portion that is a helical or otherwise non-linear structure. Advantageously, a non-linear electrode structure, such as a helix, provides additional stability when the electrode is incorporated into a nerve. Further, such a structure of an electrode provides multiplanar potential contact points for ingrowing fibro-axonal tissue, and a greater recording surface and a higher chance to contact axons.

The electrode 34 may be made from Pt—Ir wire (e.g., diameter 0.05 mm), shaped into a 1 cm long coil for example (diameter approx. 0.85 mm). As shown in FIG. 7, the electrode 34 may include, or be connected to, a linear wire 36 (which may be rigid or flexible) for connecting the electrode 34 to circuitry, which may be housed in the base 12 as noted above.

While the electrode material may be advantageously made of proven biocompatible materials such as Pt and Pt—Ir, it may be substituted by other materials such as stainless steel or tungsten commonly used in neural recording, carbon fibers and carbon nanotubes for flexibility and impedance, or eutectic gallium for flexibility and stretchability.

Advantageously, the electrode 34 is positioned within the channel 19 such that it is spaced, at least partly along its length, from the walls of the channel 19, for example by up to 0.4 mm. This contrasts with previously known configurations which incorporate electrodes into the walls of the neural interface. By spacing the electrode 34 from the channel walls, it is more likely to contact axons sandwiched in fibro-collagen or fibro-axonal tissue (since an electrode incorporated into the wall cannot penetrate through the fibroblasts). As mentioned previously, fibroblasts grow as fibro-collagenous layers on the surfaces of channel 19 first. As such, the spacing of the electrode 34 leaves room for the axons which subsequently grow on the fibro-collagenous layers of fibroblasts to contact with the electrode 34.

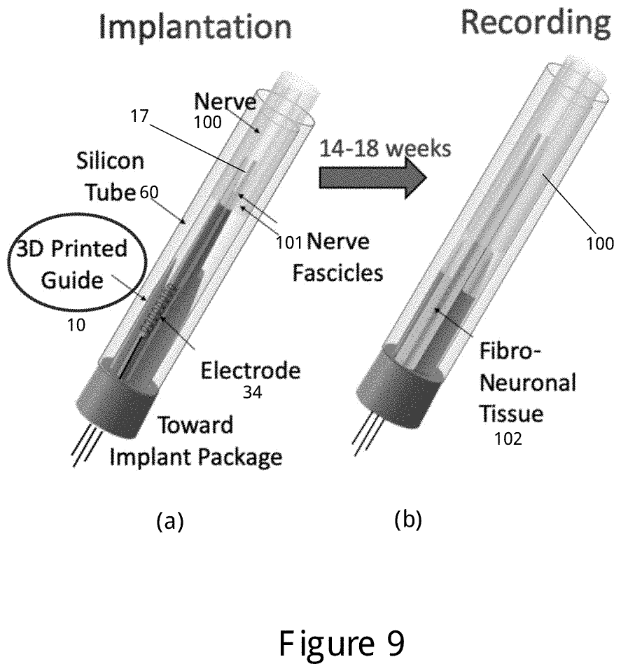

Turning now to FIG. 9, implantation of the neural guide element 10 in a nerve 100 is depicted in schematic form. In FIG. 9(a), the spire 20 of fibro-axonal guide element 10 is shown housed in a silicone (or other synthetic material) tube or sleeve 60, and the tapered end 17 of needle structure 16 is inserted into a fascicle 101 or other part of the nerve 100, sliding sleeve 60 over the nerve 100 to guide the tapered end 17 into the fascicle 101, or into the other part of nerve 100. After a variable period of time allowing for regrowth and maturation of the fibro-neuronal or fibro-axonal tissue, as shown in FIG. 9(b), fibro-axonal/fibro-neuronal tissue 102 grows along and encapsulates the needle structure 16 into the channels 19 of the guide element 10, guided by the elongate groove structures 16 and 30, and contacts electrode 34 to form an electrical connection as part of a stable, long-lasting neural interface between the guide element 10 and the nerve 100.

A number of alternative structures for the guide element 10 are possible. For example, as shown in FIG. 10, an alternative embodiment of a guide element/core 40 may take the form of a rod of substantially constant cross-section. As seen in the cross-sectional view of FIG. 10, the outer surface of the rod carries a plurality of grooved structures 42, each having a plurality of grooves (here shown having a sawtooth formation, but other shapes, for example having non-inclined sidewalls, may also be used). The grooves are triangular in cross-section, but are elongate and extend along the length of the guide element 40.

The grooved structures 42 are interspersed with non-grooved, cylindrical portions 44. It will be appreciated, though, that such smooth cylindrical portions 44 are not necessary, and that the entire outer surface of the rod/core 40 may have elongate grooves 42 disposed thereon, for example as shown for the guide element 50 in FIG. 11. In the embodiment of FIG. 11, the guide element 50 is hollow, having an inner void 52, but it will be appreciated that guide element 50 may also be a solid structure.

In the embodiment presented in FIG. 10, the grooves 42 are triangular in cross-section, but the grooves may have other shapes, for example, having rectangular cross-sections or circular cross-sections with longitudinal channels. Similarly, the grooves 30 of the guide element 10 of FIGS. 1 to 8 may have a non-triangular cross-sectional shape, such as rectangular, arcuate, etc.

As mentioned, the guide element 40 or the guide element 50 may have a substantially constant cross-section. In some embodiments, however, one or both ends of the guide element 40 or the guide element 50 may be tapered, to facilitate insertion into a nerve during implantation.

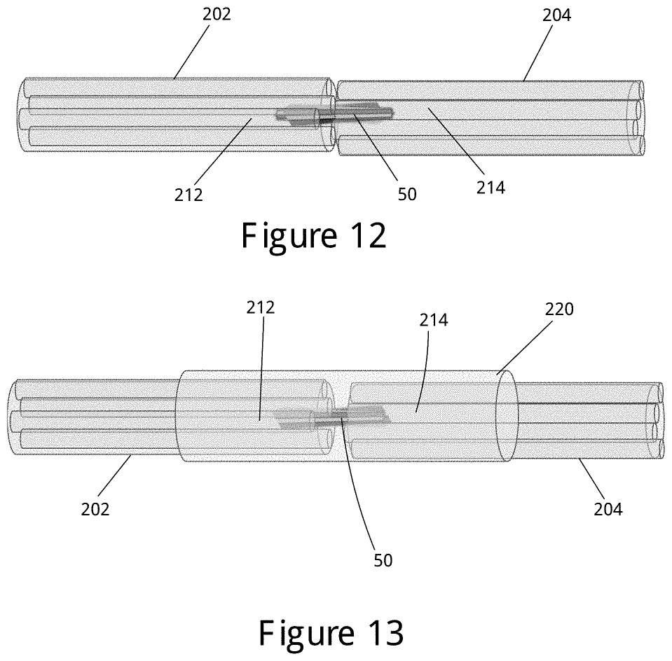

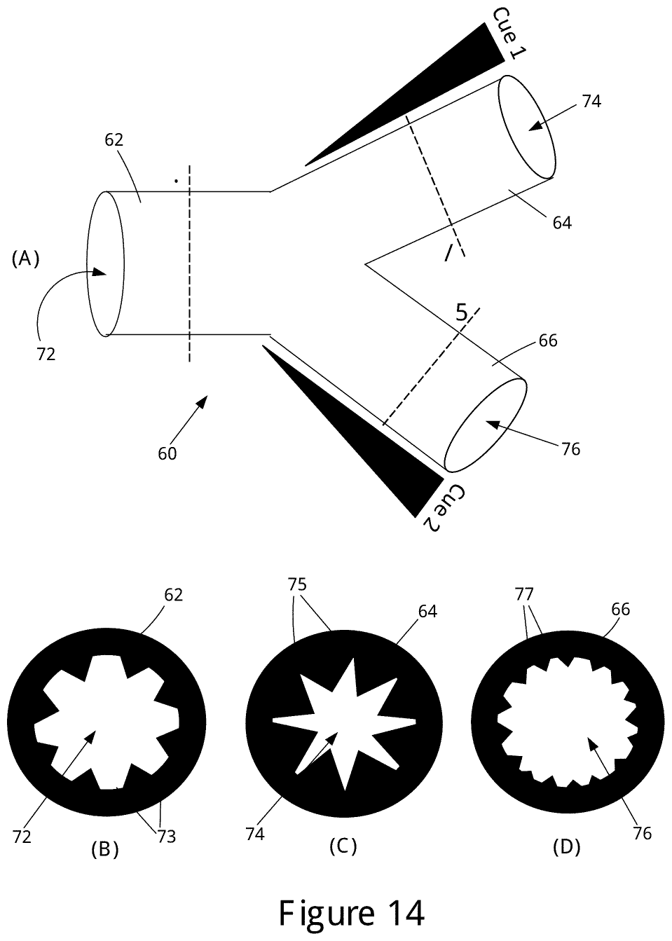

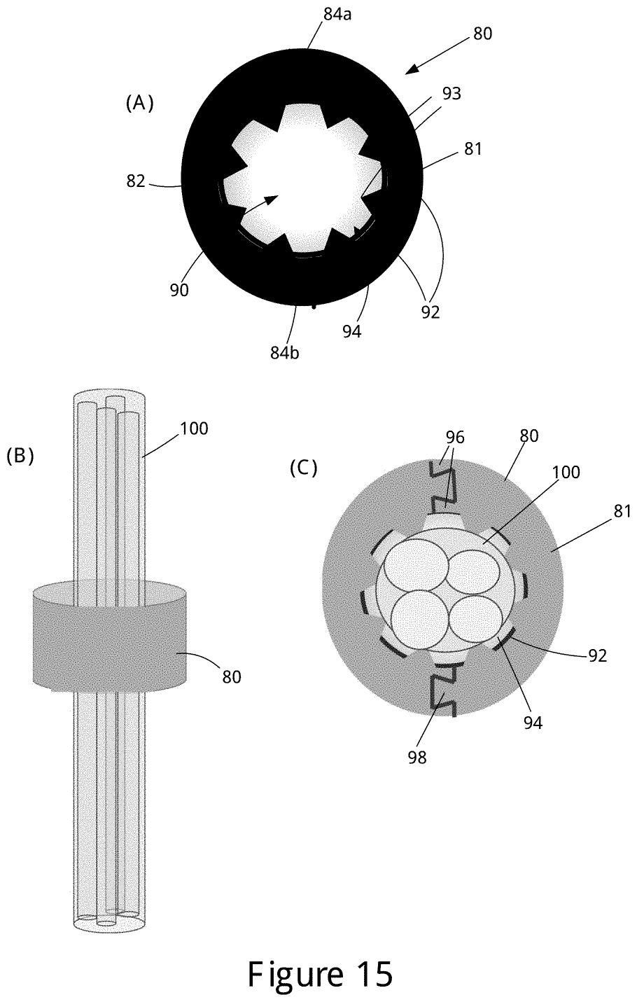

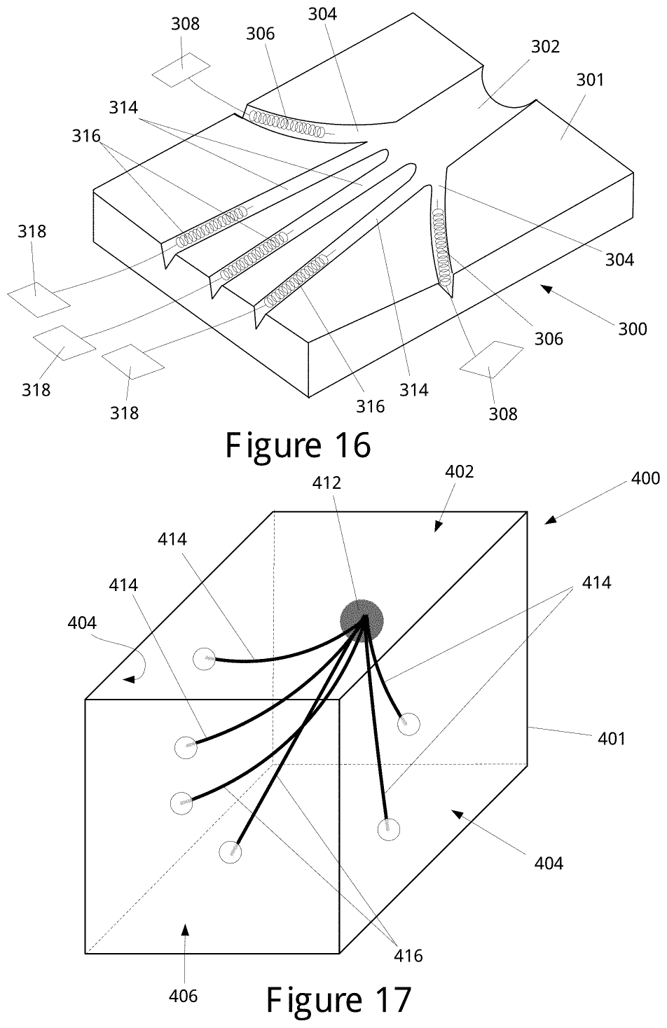

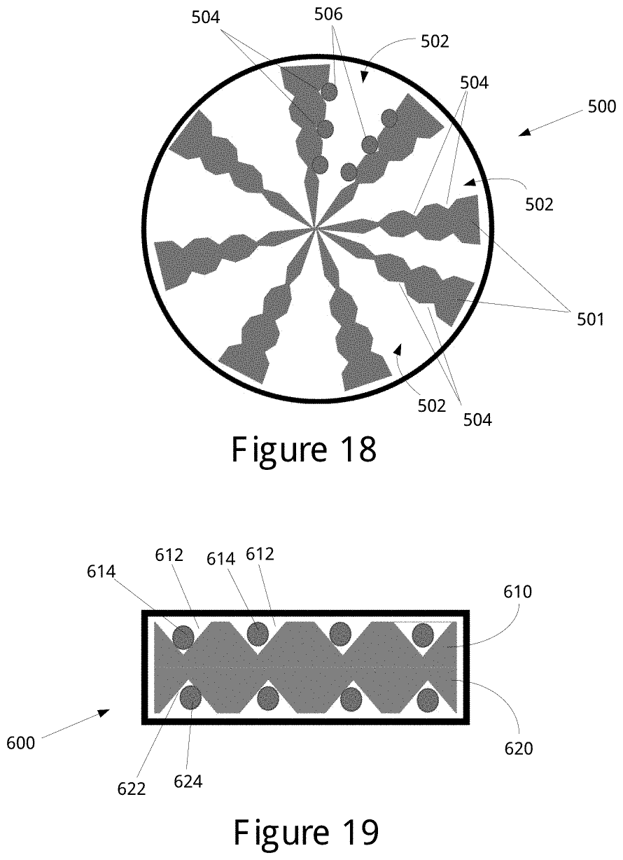

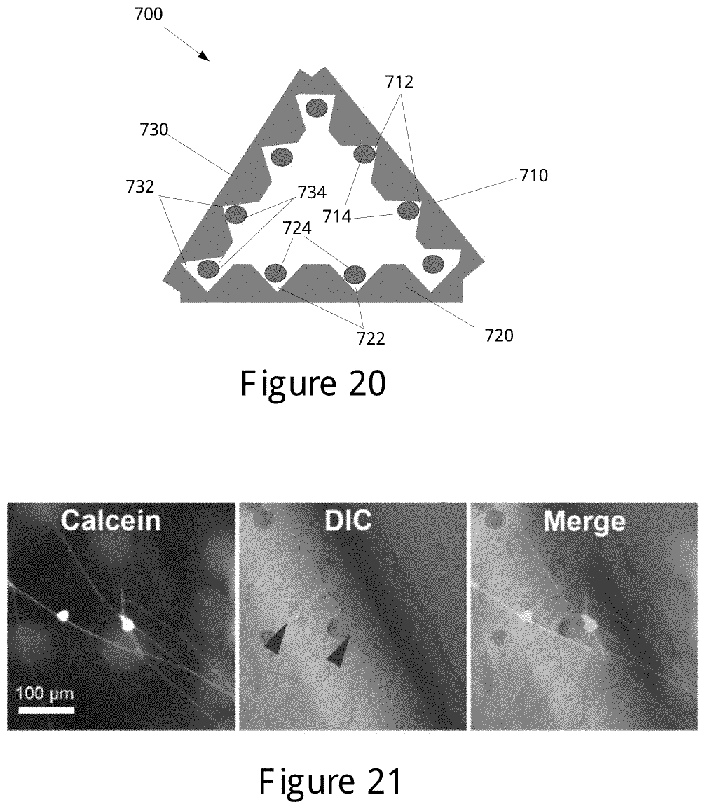

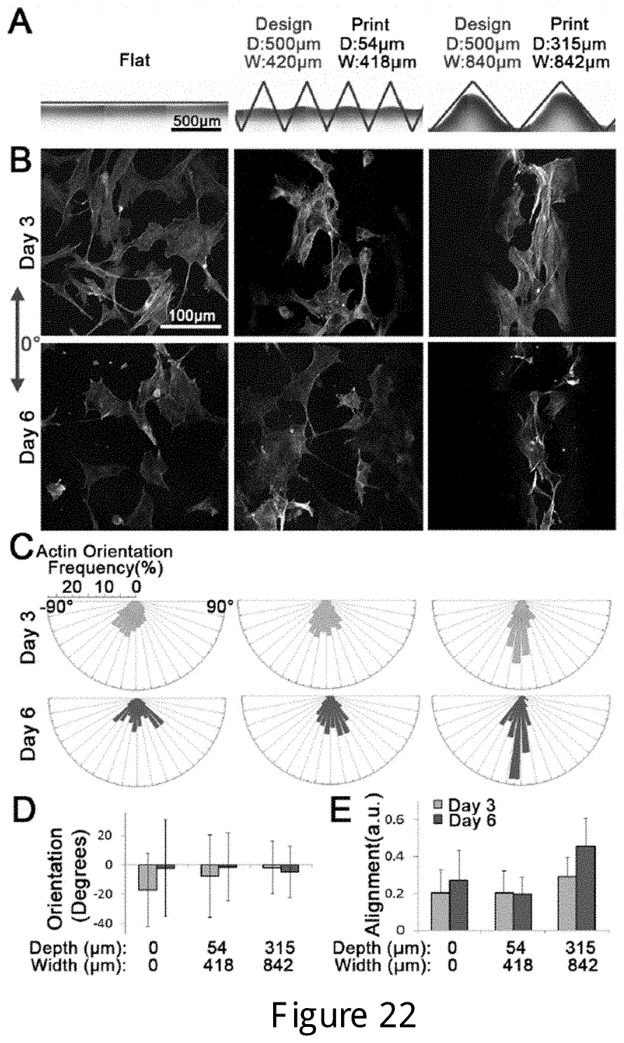

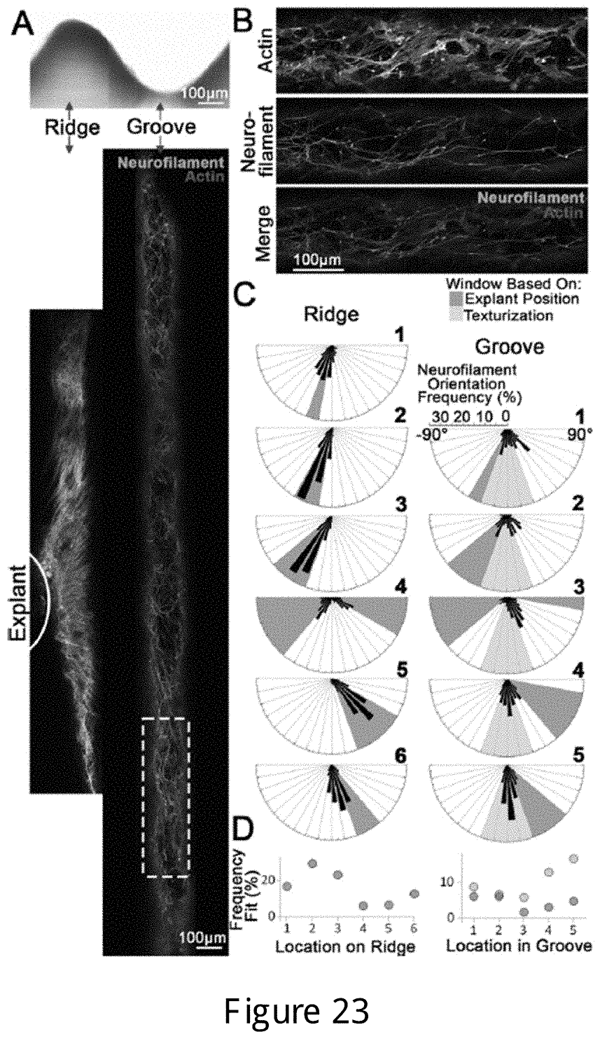

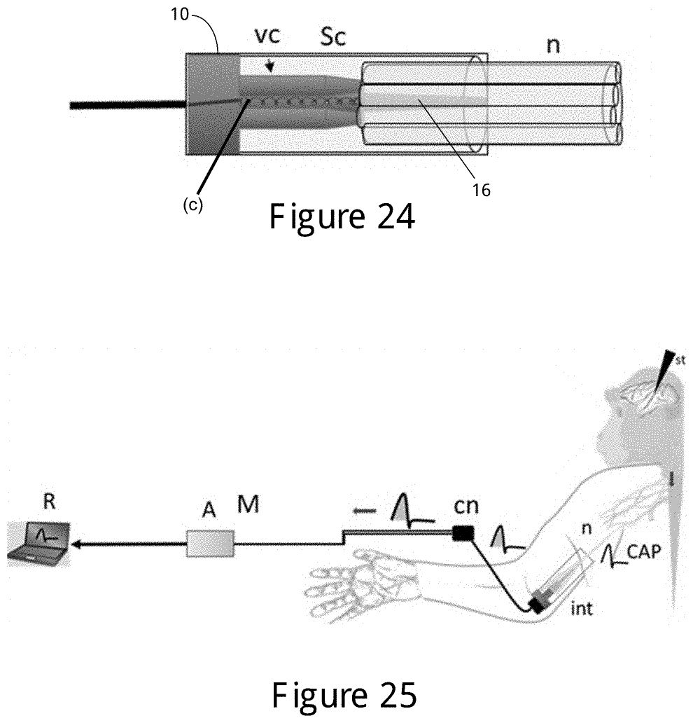

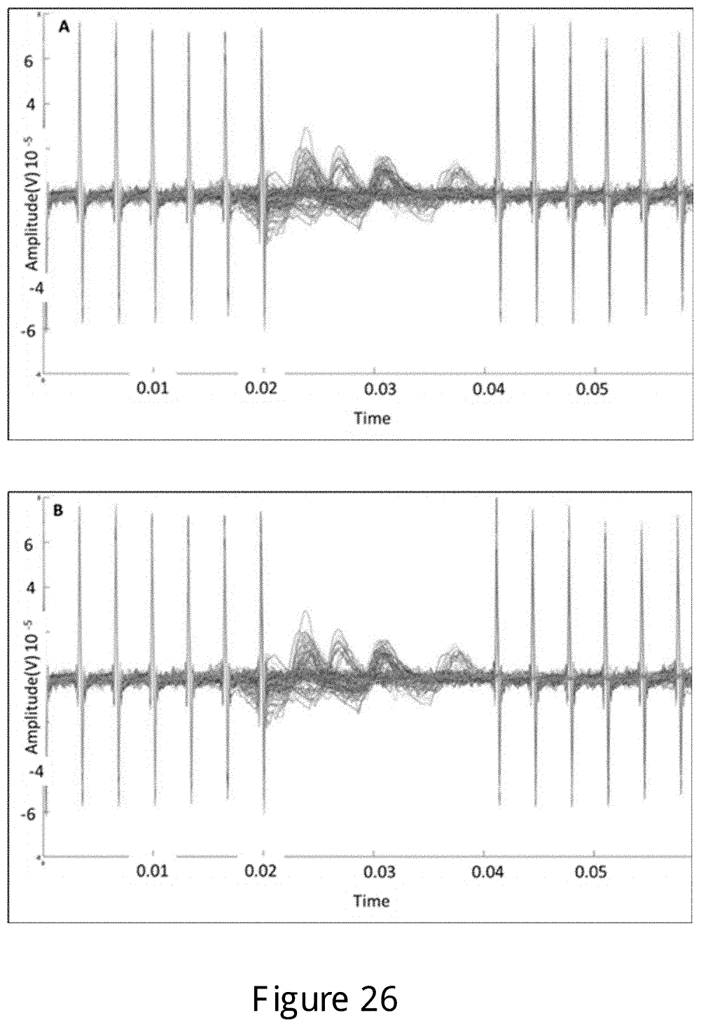

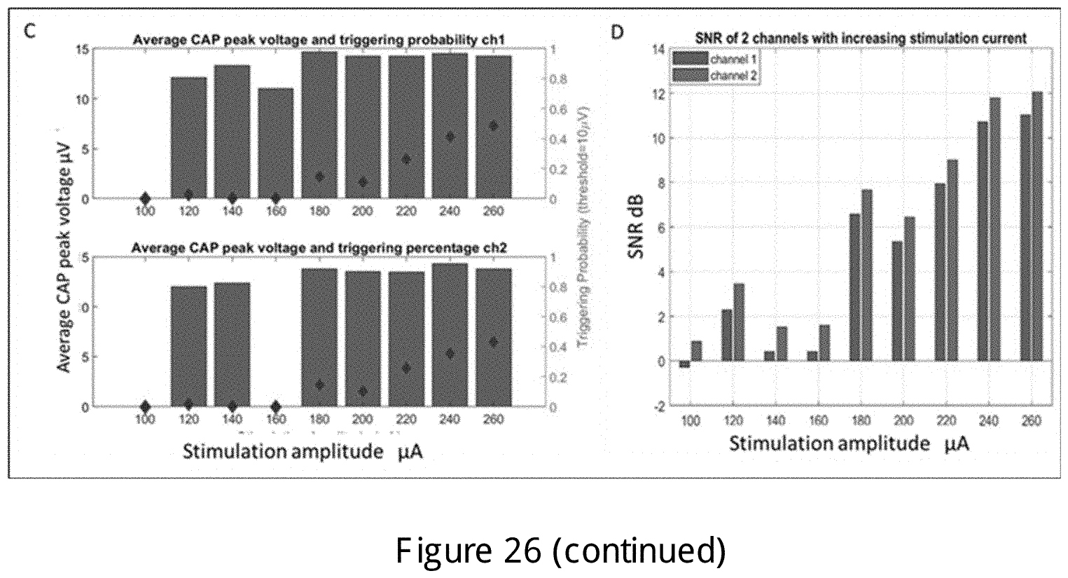

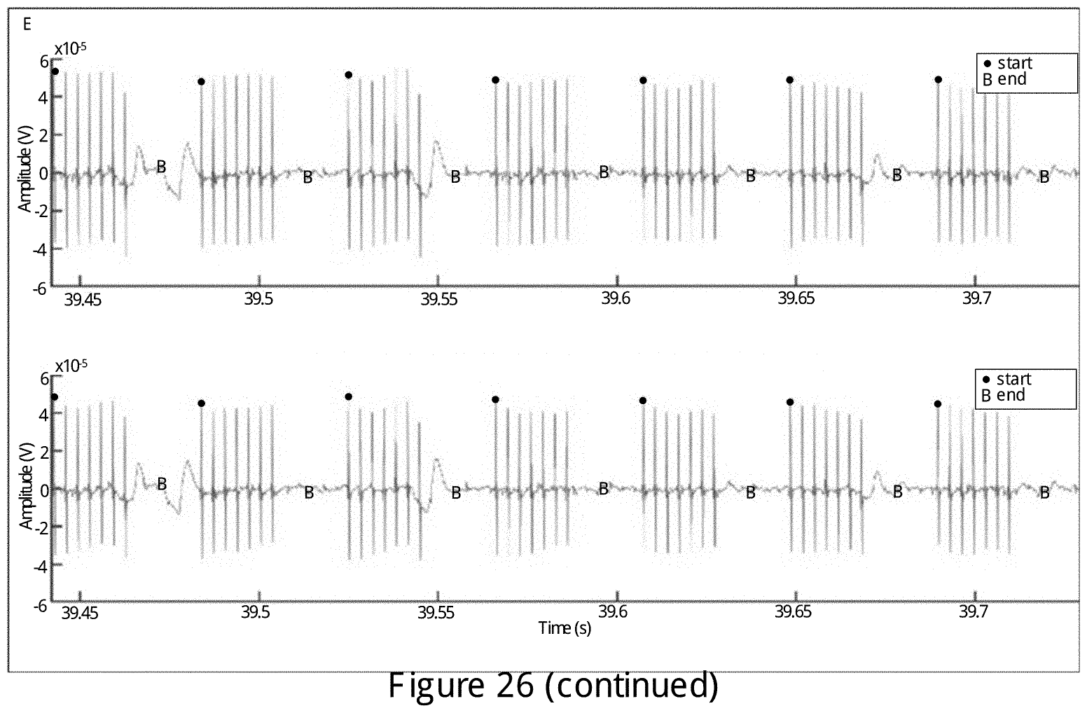

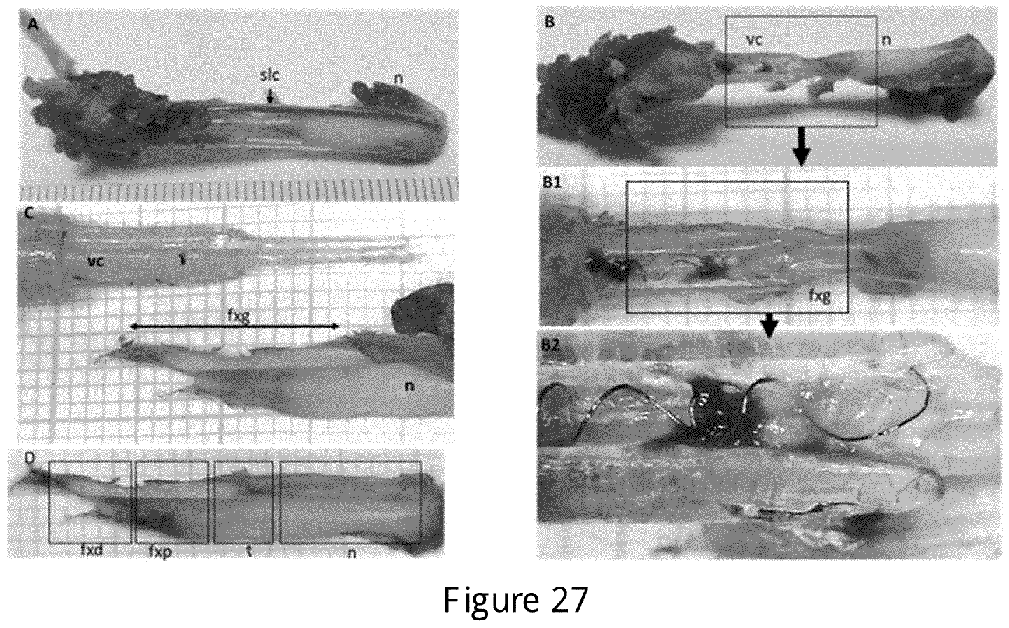

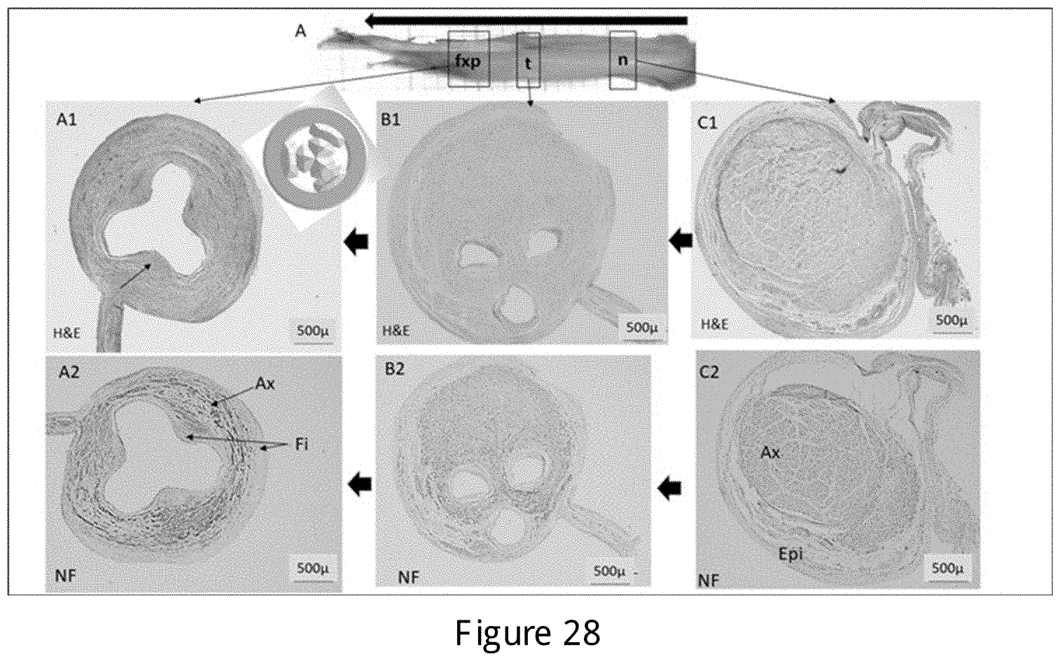

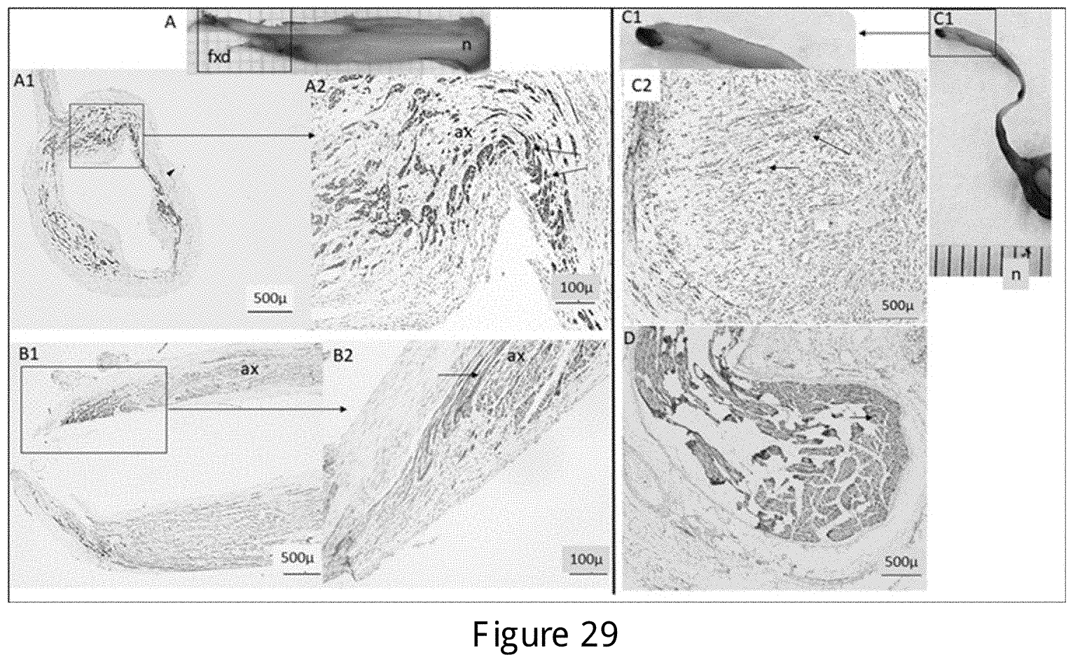

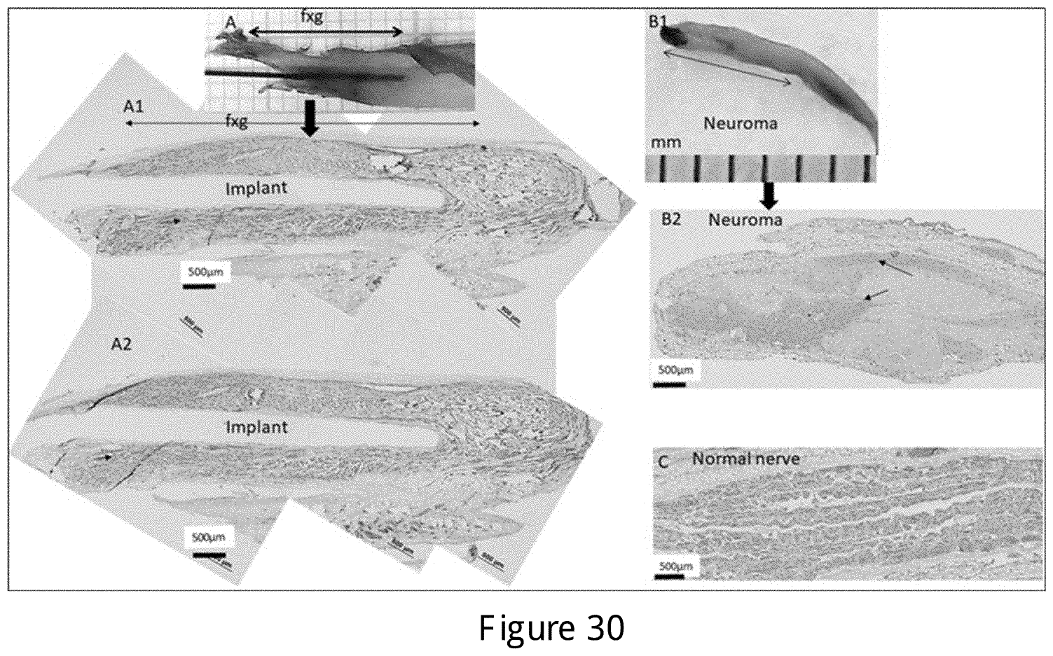

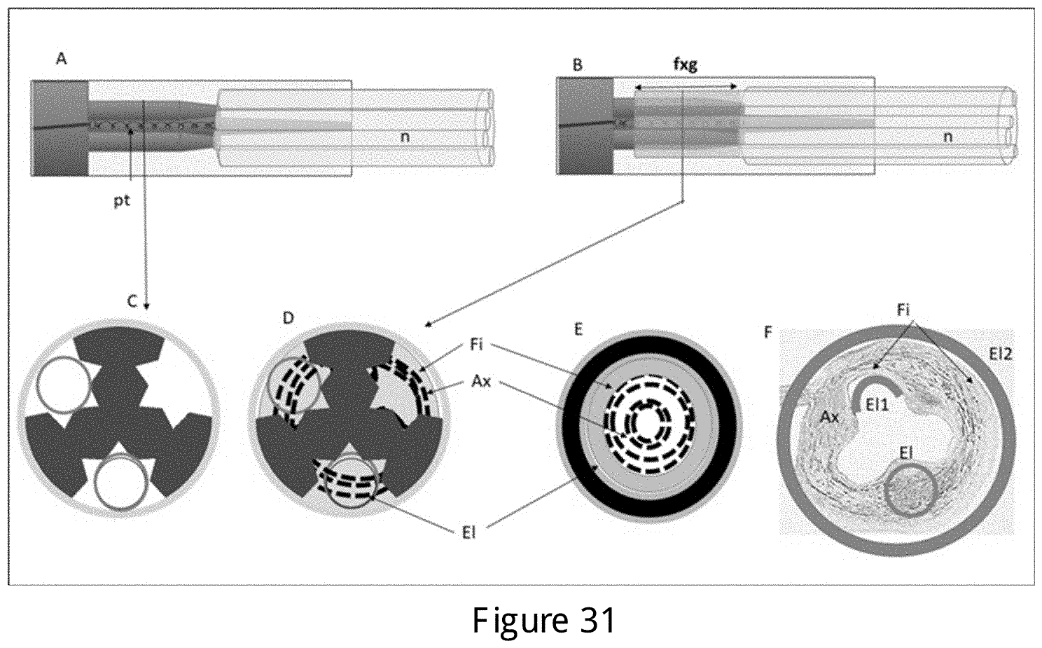

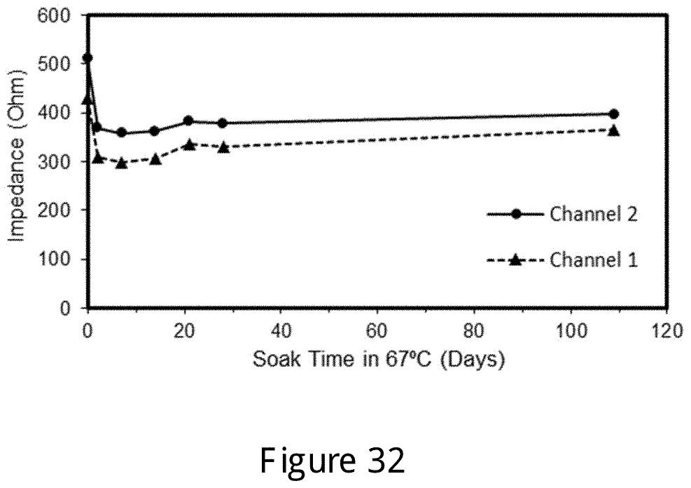

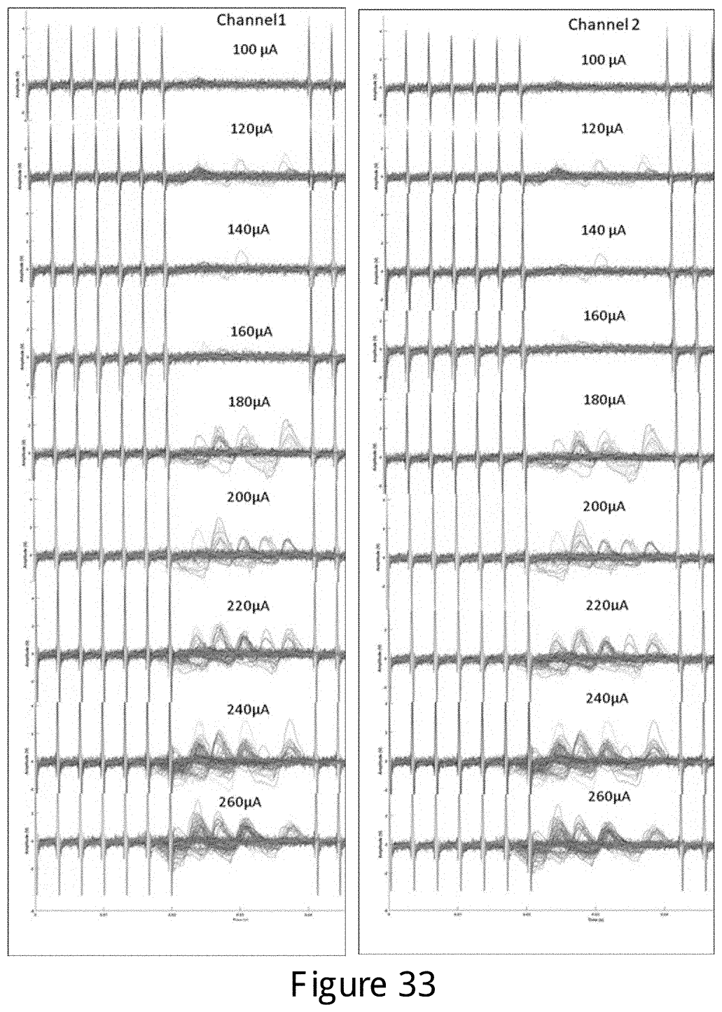

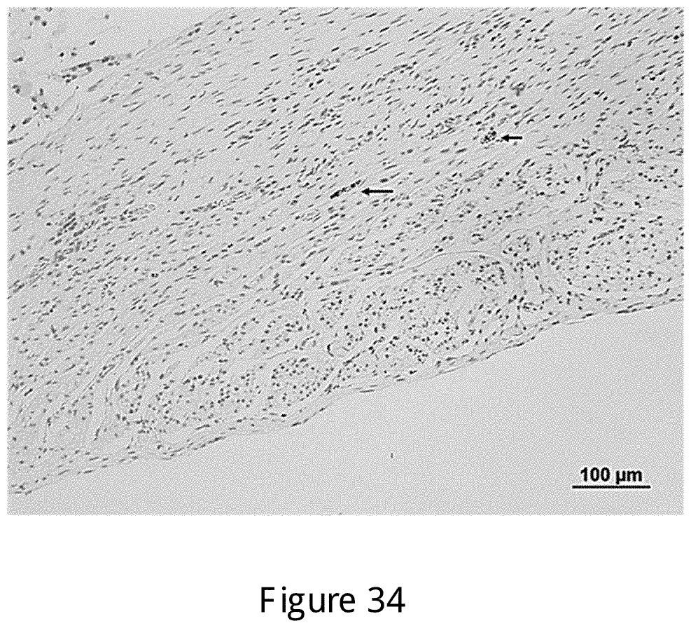

The guide element 40 or 50 may be implanted in a subject, for example for treating an injured nerve. For example, as shown in FIG. 12, opposed ends of guide element 50 may respectively be inserted into a fascicle 212 of a first nerve portion 202, and a fascicle 214 of a second nerve portion 204, the first and second nerve portions 202 and 204 being of an injured nerve which may be partially or completely severed. In the example of FIG. 12, there is a relatively small gap between the nerve portions 202 and 204, such that the guide element 50 can be used for nerve repair without additional support (e.g., sutures). If the gap is sufficiently large, a coaptation sleeve 220 may be employed in conjunction with guide element 50, as shown in FIG. 13.

Embodiments of the present invention may find application in a number of different areas. For example, neural guide elements according to certain embodiments may be used as implants in animals and in humans, for research and treatment purposes. In general, neural guide elements according to various embodiments may be suitable for neural applications that benefit from accelerating, supporting and stabilising nerve outgrowth in a specific position, such as a neural interface with electronics (e.g., stimulating and/or recording electrodes that do not cause damage to the nerve and having multiple recording/stimulating channels); directing severed nerves toward a new synapse target for targeted reinnervation; or bridging severed nerves. Further, embodiments can be used for various neural applications that require a stable position for an implant interfacing the nerve, such as for prolonged, targeted release of biomolecules at a specific neural location, e.g., for healing; or for application of physical stimulation, such as electrical, magnetic, optical, optogenetic, or mechanical stimulation of the nerve.

Embodiments of the invention may advantageously be used in one or more of the following applications:
Abiotic and biotic interface
Platform for interfacing to a nerve for signal recording
Platform for interfacing to a nerve for stimulation by a variety of means including electrical, optical, electromagnetic, magnetic and pharmacological
Device for delivering drugs to the nerve for minimizing inflammation, recover from injury, promote regeneration and repair its integrity
Device for delivering cells, such as stem cells, Schwann cells, etc to minimizing inflammation, recover from injury, promote regeneration and repair its integrity

In some embodiments, the grooves 30 or 42 may be coated with biologically active materials to promote adhesion as well as the axonal growth and regeneration. As will be appreciated by those skilled in the art, surface materials such as laminin, polylysine, fibronectin, etc. promote cell adhesion and can be beneficially used in embodiments of the present invention to provide the anchoring of the growing collagen and/or axonal fibers.

The surface coating is not limited to uniform coating. As is known in the biological disciplines, neurons are responsive to gradients of certain chemo-attracting and repelling molecules, such as netrin, semaphorin, etc. By coating the grooves 30 with gradients of these molecules, it is possible to further enhance and guide axonal growth in the grooves 30.

In yet further embodiments, cells or tissue can be incorporated on the surface of the grooves 30 or 42. Endothelial cell lining along the grooves 30 or 42 can provide the cellular foundation to the growth of the axons. Cells not only provide the adhesion to the conduit, but also the growth surface, nutrient transport, to the axons. The cellular or tissue coating may comprise diverse supporting cells, including but not limited to Schwann cells or oligodendrocytes which provide myelination to the axons and accordingly, enhanced conduction of the axonal activity.

In some embodiments, some grooves or sets of grooves (e.g., a set of grooves forming a grooved surface structure such as the internal wall of channel 19 of FIGS. 1 to 5) may have different configuration than other grooves or sets of grooves. For example, the grooves may vary in depth, width, surface coating, surface texturing (e.g., having bumps, dimples, smaller scale ridges/grooves etc. along the groove surface). Varying the groove configuration in this way may enable different types of nerve (or other) cells to grow selectively along different grooves such that those different types of cell are separately addressable (for example, for measurement or stimulation purposes). As will be appreciated by the skilled addressee, modifications of the contours and surfaces of the implantable guide can create desired shapes and configurations of fibro-axonal tissue for different applications.

In any of the embodiments described with reference to FIGS. 1 to 13, the grooves 30, 42 need not have a straight line configuration. For example, the grooves 30, 42 may comprise one or more bends, a mixture of straight line and curved segments, a serpentine configuration, and the like. In some embodiments, a groove may fork into a plurality of secondary grooves. The secondary grooves may vary in configuration from each other. For example, some secondary grooves may have different depth, width, surface coating, surface texture, etc. than other secondary grooves, to enable different types of nerve (or other) cells to grow selectively along different secondary grooves.

Turning now to FIG. 14, a further example of an implantable guide element 60 will be described. The guide element 60 has a main section 62 which bifurcates into a first branch 64 and a second branch 66. Guide element 60 is suitable for selective guidance of nerve fibres based on the type of nerve fibre, by providing different texturisation (topographical cues) and molecular guidance cues (Cue 1 and Cue 2) in the two branches 64, 66.

As shown in the cross-sectional view of FIG. 14(B), the main section 62 comprises an internal channel/core 72 having a grooved surface structure comprising a plurality of grooves 73 that extend around the perimeter of the internal channel 72. For example, the grooves 73 may be substantially trapezoidal or rectangular when viewed in cross-section.

The channel 72 of main section 62 opens into, and is in communication with, a channel 74 of the first branch 64. As shown in FIG. 14(C), the channel 74 of first branch 64 has a grooved surface structure comprising a plurality of grooves 75 that extend around the perimeter of the channel 74. The grooves 75 of channel 74 are shaped differently than the grooves 73 of channel 72. For example, the grooves 75 may be substantially triangular when viewed in cross-section. Additionally, the grooves 75 may have different depth, width and/or pitch compared to the grooves 73.

The channel 72 of main section 62 also opens into, and is in communication with, a channel 76 of the second branch 66. As shown in FIG. 14(D), the channel 76 of second branch 66 has a grooved surface structure comprising a plurality of grooves 77 that extend around the perimeter of the channel 76. The grooves 77 of channel 76 are shaped differently than the grooves 75 of channel 74 of first branch 64. For example, the grooves 77 may be substantially trapezoidal or rectangular when viewed in cross-section, as for the grooves 73 of channel 72 of the main section 62. Additionally, the grooves 77 may have different depth, width and/or pitch compared to the grooves 75 of the first branch 64. Further, the grooves 77 may have different depth, width and/or pitch compared to the grooves 73 of the channel 72 of the main section 62.

In addition to the differences in surface structure, first branch 64 and second branch 66 may differ in terms of surface coatings (such as compositions including growth factors, cell adhesion promoters, etc.) that are applied to the respective grooves 75 and 77. Together, the different surface texturisation and coatings may be arranged to selectively enable different types of cells to adhere and different types of nerve fibres to grow over and encapsulate each channel 74, 76, for example small v. big, motor v. sensory, myelinated v. unmyelinated, etc.

It will be appreciated that many variants of the guide element 60 of FIG. 14 are possible. For example, the guide element 60 may have a main body that is generally cylindrical, or otherwise generally uniform in cross-section, and the channels 72, 74, 76 may all be internal to the main body. Further, in some embodiments, the main section 62 may have a tapered end to facilitate insertion into a tube or other like structure (such as coaptation tube 220 of FIG. 13) for connection of the guide element 60 to a nerve.

Turning now to FIG. 15(A), a yet further embodiment of a guide element 80 is shown. The guide element 80 may be deployed with an intact nerve 100, as shown in FIG. 15(B).

Guide element 80 may be of generally cylindrical shape as depicted, but other external shapes are possible, for example spherical, oblate spheroid, ellipsoid, etc. The guide element 80 has a generally cylindrical internal structure 90 having a grooved structure on its internal surface, the grooved structure comprising a plurality of grooves 92 that are interleaved with a plurality of ridges 93. The grooves 92 extend along a longitudinal axis of the guide element 80, i.e., in a direction that is generally aligned with nerve 100 when the guide element 80 is attached to it.

The guide element 80 may comprise a first portion 81 adapted to couple with a second portion 82 to enclose the nerve 100, as depicted in cross-section in FIG. 15(C). In one embodiment, a pair of snap-fit connections 84a, 84b is used to connect the two portions 81 and 82. To this end, each portion 81 or 82 may carry, on one side, a pair of cantilever arms 96, and on the other side, an outwardly flared locking element 98, as illustrated for the first portion 81 of the guide element 100 in FIG. 15(C). Accordingly, the two portions 81, 82 are complementary to each other in that a locking element 98 of one portion 81 may be inserted between cantilever arms 96 of the other portion 82 to deflect them to effect the snap-fit connection (and vice versa). It will be appreciated that many other forms of snap-fit connection are possible, though the connection shown in FIGS. 15(B) and 15(C) is advantageous in that it provides for a smooth join between the portions 81 and 82.

In some embodiments, only a single snap-fit connection (e.g., 84a) may be needed, with a hinge or like structure being provided in place of the other snap-fit connection (e.g., 84b).

The use of a snap-fit connection to enclose the nerve 100 may ensure that no nerve damage is caused, as no significant prolonged compression on the external surface of the nerve occurs. To this end, the diameter of the channel 90 may be designed such that the ridges 93 of the grooved surface structure do not penetrate into the nerve 100.

It will be appreciated that, in some embodiments, the guide element 80 need not entirely encompass the nerve 100. For example, the guide element 80 may be C-shaped in cross-section, any may comprise resilient arms and/or a hinge to enable the guide element 80 to be “clipped” around nerve 100 without causing nerve damage.

The grooves 92 of the guide element 80 support adhesion to the nerve and stop the guide element 80 from sliding up and down on the nerve 100. To this end, the grooves 92 may optionally carry a surface coating that contains a cell adhesion promoter.

The guide element 80 may contain, within grooves 90, one or more components for delivering one or more stimuli to nerve 100, or to record signals travelling along the nerve 100.

In one example, one or more of the grooves 92 may have a surface coating containing one or more drug compositions that are released and absorbed into the nerve 100 when the guide element 80 is attached to the nerve 100, as shown in FIG. 15(C).

In another example, an electrode (such as a helical or part-helical electrode as depicted in FIG. 7) may be disposed within channel 90 for interfacing the nerve 100 to electrical components that are external to the channel 90 (for example, being housed in an external part of the guide element 80). The external electrical components may then be used to record signals from nerve 100, and/or to deliver a stimulus to the nerve 100 (e.g., to transmit control signals along the nerve). In some examples, the electrode may be used as a heating element to deliver thermal energy to nerve 100.

A further example of a multichannel guide element 300 is shown in FIG. 16. The multichannel guide element 300 may be used for recording of signals from a nerve. Guide element 300 has a substrate 301 in which is formed a first channel 302 and a plurality of secondary channels 304 and 314 branching from the first channel 302. Each of the secondary channels 304, 314 may have surface texturing comprising a plurality of grooves, as in any of the embodiments of FIG. 1-8, 10-11, or 14. The secondary channels 304, 314 branch away from the first channel 302 in a plane of the substrate 301 for two-dimensional guidance of fibro-axonal growth from the end of a nerve located in first channel 302 into the secondary channels 304, 314.

Each secondary channel 304, 314 carries a respective helical electrode 306, 316, which is in turn connected to a respective transducer 308, 318. As fibro-axonal tissue grows within a channel 304, the windings of helical electrode 306 maintain contact with axons in the fibro-axonal composite, despite the presence of fibrotic tissue, such that transducer 308 can still record signals conducted along the axons (and likewise for channels 314, electrodes 316, and transducers 318).

Different surface texturisation and/or coatings may be applied to different channels 304, 314, as discussed above.

As shown, three of the secondary channels 314 extend in a direction substantially parallel to the first channel 302, while two channels 304 extend laterally towards the sides of the substrate 301. Accordingly, with this branching configuration, greater separation of nerve fibres can be achieved, enabling greater ability to record neural activity via finer access to specific locations of the nerve, and also more easily enabling placement of transducers 308, 318 at varying locations and orientations. Further, by providing a planar configuration, the guide element 300 creates a flat layer of fibroaxonal tissue, which may be important for satisfying anatomical constraints in some applications.

FIG. 17 shows another example of a multichannel guide element 400. The guide element 400 extends from a first end 402 to a second end 406 and has at least one sidewall 404. As shown, the guide element 400 is a rectangular prism, but many other shapes are also possible. The guide element 400 has a body within which extend a plurality of channels 414, 416. Each channel 414, 416 may carry an electrode (not shown). Some of the channels 416 extend towards the second end 406, while others extend towards the sidewalls 404. Accordingly, the channels 414, 416 provide a network that stimulates growth of fibro-axonal tissue from a nerve that is placed at an entry 412 of the guide element 400, in a plurality of directions and orientations in similar fashion to the guide element 300, but in three dimensions rather than two.

FIGS. 18 to 20 show three further alternative configurations of guide element.

FIG. 18 shows a cross-sectional view of a guide element 500 that has a plurality of channels 502 that are defined between grooved structures 501 that extend from a centre of the guide 500 towards its periphery. Each grooved structure 501 has a plurality of grooves 504 on opposed surfaces thereof. A plurality of these grooves 504 in each channel 502 may each have a helical electrode 506 located therein, which maintains contact with axons of fibro-axonal tissue as the fibro-axonal tissue grows within the channel 502. By providing multiple electrodes it is possible to obtain more fine-grained signal measurements in each channel 502.

FIG. 19 shows a cross-sectional view of a guide element 600 that has a first planar layer 610 and a second planar layer 620 opposite the first planar layer 610. First planar layer 610 has a plurality of grooves 612, and a plurality of helical electrodes 614 located in the grooves 612. Second planar layer 620 has a plurality of grooves 622, and a plurality of helical electrodes 624 located in the grooves 622. Advantageously, with this configuration, flat two layer fibro-axonal growth may be generated.

FIG. 20 shows a cross-sectional view of a guide element 700 that is in a triangular configuration with a first planar layer 710, a second planar layer 720, and a third planar layer 730, the layers 710, 720 and 730 forming the sides of the triangle. First planar layer 710 has a plurality of grooves 712, and a plurality of helical electrodes 714 located in the grooves 712. Second planar layer 720 has a plurality of grooves 722, and a plurality of helical electrodes 724 located in the grooves 722. Third planar layer 730 has a plurality of grooves 732, and a plurality of helical electrodes 734 located in the grooves 732. By configuring the guidance channel with multiple surfaces in this way, it is possible to adapt the fibro-axonal growth to multiplanar placement of transducers. It will be appreciated that other shapes are also possible, such as square, pentagonal, etc.

Any of the embodiments above may be used for therapeutic or research purposes. An example of treatment with the guide is directing axonal growth towards a synaptic target (biotic or abiotic) to limit neuroma-related pain. A biological target can be a muscle tissue. The guide could provide an optimum neuroma morphology to increase the chance of axons coming in contact with muscle fibres and forming neuromuscular junctions which in turn limits the neuroma related pain.

Experimental data demonstrating various aspects of certain embodiments will now be described with reference to FIGS. 21 to 34.

### Experimental Studies

In Vitro Experiments

Methods

A. Substrate Preparation and Characterization

1×1 cm substrates were 3D printed in VeroClear RGD810 with Objet260 Connex3 (Stratasys, Singapore) according to a design prepared in SolidWorks Software. Upon removal of the scaffold material and cleaning with isopropanol and phosphate buffer saline (PBS) the substrates were coated with an approx. 2 nm-thick layer of Parylene C. The substrates were imaged with a light microscope and their geometry was quantified in ImageJ software. Prior to cell plating the substrates were sterilized by 70% ethanol and 30 min-long UV exposure.

B. Cell Culture

Mouse NIH3T3 fibroblasts were plated on the VeroClear substrates at 15×103 cell cm-2 density and were cultured with DMEM media supplemented with 1% penicillin-streptomycin and 10% Fetal Bovine Serum.

Dorsal Root Ganglion neurons (DRGs) were obtained from embryonic day 14 rats as described in H. U. Lee et al., “Subcellular electrical stimulation of neurons enhances the myelination of axons by oligodendrocytes,” PLoS ONE, vol. 12, July 2017, Art. no. e0179642. Dissociated DRGs were plated on the VeroClear substrates at 25×103 cell cm-2 density. Neuron culture and imaging with Calcein AM was performed as described in Lee et al.

DRG explants were plated on top of the fibroblast layer by lowering media level and gently placing individual explants on top of the fibroblasts. Fibroblast culture medium was supplemented with 100 ng mL-1 NGF and 2% B27 to support neuronal growth. The fibro-neuronal co-culture was maintained by half media exchange every second day.

C. Immunostaining

Fibroblast cultures were fixed by 15 min-long incubation with 4% paraformaldehyde (PFA) in PBS. Following 3×5 min washes with PBS the cells were permeabilized with 0.3% (w/v) Triton X-100 for 5 min and incubated with AlexaFluor 568 Phalloidin (1:100) for 1 h. The substrates were mounted on glass coverslips with ProLong antifade.

Fibro-neuronal co-cultures were fixed by 60 min-long incubation with 4% PFA in PBS. Following 3×5 min washes with PBS the cells were exposed for 2 h to blocking solution: 0.3% (w/v) Triton X-100 with 5% bovine serum albumin in PBS. Primary mouse antibody against neurofilament (1:500) was applied overnight at 4° C. Following 3×5 min washes with PBS the cells were incubated with AlexaFluor 568 Phalloidin (1:100) and AlexaFluor 488 goat anti-mouse secondary antibody (1:500) for 1 h. The cells were then washed 3×5 min with PBS and the substrates were carefully mounted on glass coverslips with ProLong antifade.

D. Imaging and Analysis

All images were taken with an inverted Zeiss LSM 800 Microscope controlled with Zen Blue Edition software. The VeroClear substrates were imaged in DIC with 10× objective. Imaging of neurons for testing VeroClear biocompatibility additionally used green channel with 488 nm light wavelength.

Fibroblasts and fibro-neuronal co-cultures were imaged in the confocal laser scanning mode with long distance 20× objective. The laser was used at 561 nm and 488 nm for visualization of AlexaFluor 568 (red), AlexaFluor 488 (green) channels, respectively. For fibro-neuronal co-cultures, the channels z-dimension was offset by 6-10 μm to separate actin staining of non-neuronal and neuronal cytoskeleton. ImageJ software was used to analyze all images. Actin and neurofilament alignment were measured with OrientationJ plugin, which evaluates an orientation for each pixel based on the structure tensor. A histogram of orientations was generated by the Orientation 3 Distribution tool. Each histogram ranged from −90° to 90°, where angle 0° corresponds to the groove direction (textured substrates) or printing direction (flat substrate). To minimize the effects of background noise and the out of focus actin filaments, the cut off energy and coherency were set as 5% and 40%, respectively. The degree with the highest orientation frequency was used as the image's orientation. The frequencies were normalized to the total area under the histograms, averaged for multiple images (for actin) and collated into 10°-wide categories. For actin measurements, the percentage of the orientation frequencies in the 30° peak window (the peak category and the two adjacent categories), was used as a measure of alignment. For neurofilament measurements, each image was assigned a probability window based on the angle range of the image's location to the explant's position. For the groove, we additionally assigned a probability window (−20° to 20°) based on the texturization. Frequency fit is defined as an average orientation frequency in a window.

Results

A. VeroClear is Biocompatible for Neuronal Growth

VeroClear is a common 3D-printing photopolymer simulating acrylic. It is favoured for its low cost, ease of use and physical properties: rigidity, transparency and dimensional stability. These features are of value for biomedical studies.

However, VeroClear is a mixture of components and its exact biocompatibility is not fully tested. Nevertheless, recent studies show that VeroClear supports regular growth of microbes, and mammalian cells, like hepatocytes and endothelial cells. To test if VeroClear can be used with highly sensitive primary neurons we coated its surface with Parylene C, Poly-L-Lysine and Laminin, prior to plating embryonic DRGs.

The morphology of the cells growing on VeroClear was the same as on the control polystyrene substrate, as assessed with Calcein AM dye after 5 days of culture (see FIG. 21, in which Calcein dye (green) visualizes viable neurons after 5 days of culturing; DIC channel shows VeroClear surface; and red arrowheads point at neuronal cell bodies visible on VeroClear). Additionally, thanks to its transparency and limited autofluorescence, VeroClear was proven compatible with microscopy-based assays.

B. Fibroblasts Align with the Substrates' Grooves

Actin filaments are dynamic cytoskeletal fibers constantly restructuring themselves to facilitate cellular adherence, motion, reshaping, or intracellular transport. Accordingly, when fibroblasts sense the environment's microtopography they adjust their actin filaments in stress fibers. To test fibroblast alignment we printed a flat control substrate and two textured substrates: with mid-sized and large grooves. The side walls of the grooves were within the size achievable by fibroblasts (several hundred μm for NIH3T3). Being aware of the accuracy limitations of our printing set up, we incorporated an offset into our design. As anticipated, the groove width, but not depth, was printed according to the design (FIG. 22A). The flat surface also showed aligned grooves (depth: approx. 10 μm) due to the line-by-line printing mode. Fibroblasts were cultured on the substrates and fixed on day 3 and 6. The cells were observed growing on all areas of the substrate.

Eight to twelve, 312×312 μm images of the cells in the grooves' bottom or on the flat substrate were used for the analysis. Actin filaments that were out of focus were excluded. Through image analysis we generated histograms of averaged actin orientation frequency for each substrate (FIG. 22C). For all substrates, fibroblast actin was oriented toward 0°, that is, along the printing direction for the flat substrate and along the groove direction for the textured substrates (FIG. 22D). The highest orientation frequency peak, indicating the highest alignment, was observed for the substrate with the large grooves. This trend was also observed with the alignment index measurement (FIG. 22E).

The observed partial alignment on the flat substrate can be attributed to the minor grooves created during the printing process. The steep slope of the large grooves provided structural contact, but also, it is possible, that due to the gravitational forces, it enforced fibroblasts to grow along the grooves as it was the only available lateral direction.

FIG. 22 shows: A. Stitched images of the substrate contours with an overlay of the respective design (red line). The depth (D) and width (W) of the design (red) and the print were measured. B. Actin images of fibroblasts growing on the substrates for 3 and 6 days. Purple arrow indicates 0° orientation angle, aligned with the groove orientation. C. Distribution of actin filament orientation for each substrate. The orientation frequency for each degree was normalized, collated into 10° categories, and averaged. D. Image orientation defined as the degree with the highest orientation frequency averaged for all images. E. Image alignment index based on the percentage of the orientation frequencies in the 30° peak window (the peak category and the two adjacent categories) in the orientation frequency. Error Bar=S.D.

C. Axons in a Fibro-Neuronal Co-culture Align with the Substrate Grooves

An axon probes its surrounding with a growth cone at its tip and elongate accordingly to the cues from its microenvironment. The sensed topographic features can be as small as nano-range. Fibroblasts direct other cells through secretion of ECM proteins such as type 1 collagen. These collagen fibers serve as structural handles for elongating axons. We aimed to test axon alignment in a fibro-neuronal co-culture on the textured substrate with the large grooves (D: 315 μm, W: 842 μm). Fibroblasts were cultured for 3 days before plating of a DRG explant. After another 3 days the culture was fixed and analysed. We observed good adhesion of the explant and an extensive elongation of axons. It is plausible that increased contact surface available to the explant on the texturized substrate provided additional support.

Actin and neurofilaments (neuron specific intermediate filaments) were immunostained and imaged at the ridge adjacent to the explant border and at the groove located 420 μm away (FIGS. 23A and 23B). Through image analysis we generated histograms of neurofilament orientation frequency for each location along the ridge (six 320×320 μm images) and the groove (five 320×740 μm images). Based on the angle between the locations and the position of the explant we established a window of the expected axonal orientation if grown on a flat substrate (FIG. 23C). The explant position accurately predicted the orientation frequencies on the ridge. This was confirmed with the frequency fit index—a sum of the frequencies normalized by the window width (FIG. 23D). Lesser fit was observed for the locations in the groove. To measure the effect of the texturization, we established a window of the expected axonal orientation if aligned with the texturization (−20° to 20°). For all locations, the frequency fit was higher for the angle window based on the texturization than on the explant position (FIGS. 23C and 23D).

The observed axonal alignment is a result of a contact with aligned fibroblasts, but also with multitude of glia and other cell types introduced by the DRG explant. Multilayer, interdependent structure of various cells is a closer replication of the in-vivo condition.

FIG. 23 shows: A. Stitched image of the cellular cytoskeleton—actin (red) and neurofilament (green)—of fibroneuronal co-culture on the textured VeroClear substrate. Only cells on top of the ridge and at the bottom of the groove are in focus. The corresponding substrate contour is shown on top. Neuronal explant position is shown by an overlay. B. Two times enlargement of the region marked with the dashed line in (A). C. Distribution of neurofilament orientation frequency in six 320×320 μm locations along the ridge, and five 320×740 μm locations along the groove. The orientation frequency for each degree was normalized and collated into 10° categories. The windows of expected axon orientation based on the explant position are marked in pink. The windows indicating alignment with the texturization (−20° to 20°) are marked in gray. D. Quantification of the fit of the orientation frequencies to the windows based on the explant position (pink), and texturization (gray).

In Vivo Experiments

Materials and Methods

Study design: We have conducted a feasibility study into the surgical implantation of specifically designed implants with electrode onto the ulnar nerve of macaque subjects for determining the potential of a long-term neuro-prosthetic interface. The implants were embedded in-situ for a period of 4 months following which electrophysiologic studies and histology were performed.

Animals: Institutional guidelines and IACUC approval were obtained for the use of animals as well as the experimental protocol. 5 male macaques (Macaca fascicularis), weighing 15-20 Kgs were used. An implantable neural guide 10 (FIGS. 1-8) was placed in the ulnar nerve in three macaques. Two macaques were used as controls where the electrodes were incorporated within a silicone sleeve without the implant 10. Macaques were used only after demonstration of this concept in earlier experiments on rats and was subject to strict institutional guidelines. Division of the ulnar nerve was used as an alternative to amputation of the limb. The cut ulnar nerve simulated the nerve end that would be encountered in an above elbow amputation. Being the minor nerve in the macaque hand, it rendered minimal disability to the hand function.

Implant Fabrication: The implant 10 was 3D printed in VeroClear RGD810 (Creatz3D, Singapore) with Objet260 Connex3 (Stratasys, Singapore) according to the design prepared in SolidWorks Software. The design comprised a tapering spire 20 having a length of 20 mm. Three ridges 22 were incorporated, creating 1.34 to 2 mm-wide channels. The construct had a pedestal 12 of 5 mm diameter. Upon removal of the scaffold material and cleaning with isopropanol and phosphate buffer saline (PBS) the surfaces were coated with approx. 2 nm-thick layer of Parylene C. The electrodes were made from 2.4 cm long, stripped Pt—Ir wire (diameter 0.05 mm) shaped into a 1 cm long coil (diameter approx. 0.85 mm) and incorporated in two of the guidance channels 19, connected via crimping to a flexible coated wire (Cooner Wire USA) and connected to a 4 pin connector. The electrode/Wire crimp connection was incorporated within the implant's base 12 for mechanical stability and hermetically sealed with slow curing polydimethylsiloxane (PDMS, Dow Corning) that allowed avoiding introducing air bubbles in the direct contact with the electrode to improve its durability, and with an additional, outer layer of silicone elastomer (Kwik-Sil, World Precision Instruments) to provide additional mechanical and liquid barrier.

Durability Testing: The long-term electrical durability of the 3D construct with the electrodes was monitored via an accelerated (67° C.) soak test. The construct was placed in 10% PBS and incubated in an oven at 67° C. The relative impedance sine waveform of the two channels to the construct's reference electrode was regularly scanned across 10 Hz-30 kHz frequencies using an impedance analyser. An average of two repeated impedance measurements at 1077.5 Hz was used to monitor electrode electrical connectivity and the exposed site metal status. Prior to implantation, the impedance of the construct was monitored during 15 week-long soak tests at 67° C. In the first week, the impedance was measured twice, then weekly until the end of the first month. The last measurement was at week 15 to test long term stability (FIG. 32). Before reaching steady values, the electrode impedance showed an initial drop, attributed to metal-fluid interface equilibration. The impedance remained stable after 109 days in 67° C., which is approximated to correspond to over 2 years (872 days) in 37° C., the temperature of the human body. The results strongly indicate a long-term electrical durability of the construct.

Surgical implantation: Before implantation, the construct above the implant's base 12 was placed in a medical grade silicone cylinder 60 of a slightly smaller diameter that provided a tight fit, without a need for any additional sealing mechanism. The silicone cylinder 60 had a lateral incision for ease of nerve insertion. Prior to implantation, the complete construct was sterilized with ethylene oxide. For the control group micro-electrodes 34 were placed within the silicone cylinder 60 without the implant 10. The surgery was carried out in the operating room under general anesthesia with isoflurane and strict surgical sterility. An 8 cm incision was made on the medial aspect of the arm. The ulnar nerve was identified and divided proximal to the elbow. The proximal cut end was placed within the cylinder, and the core (c) was inserted within the inter-fascicular space (FIG. 24). The nerve with the implant 10 was buried within the intermuscular plane between the biceps and the brachialis muscles. The connector was placed in the subcutaneous tissue. The incision was closed with Vicryl® sutures and dressing was performed. Postoperatively, antibiotics and analgesia were provided for a period of 1 week. The animal was allowed free movement and use of the limb within the cage. Day night cycle and enrichment was provided.

Electrophysiology: Electrophysiologic studies were carried out under general anesthesia without using neuromuscular blocking agents. We exposed the connector without disturbing the neural interface and connected to an Intan biopotential recording system (Intan Technologies, LLC). Craniotomy was performed to expose the contralateral motor cortex. Needle stimulation was used to locate the precise region of the motor cortex resulting in activation of intrinsic muscles of the hand. Simultaneous EMG electrodes were placed in the biceps muscle adjacent to the site of implant. Stimulation was carried out at the beginning with 80 μA at 20 μV increments in stimulus trains of 5 stimuli per millisecond. The interface electrodes were connected to the Intan Amplifier system (Intan Technologies). Signals were recorded for both the channels. Signals were acquired from the electrodes using a Neutrino 2 amplifier (Neutrino Technology Co. USA). The raw signals were filtered to produce the ENG signals identified for each stimulation protocol. The observed signals were within a 2 ms-5 ms time interval. Impedance measurements indicated that both electrodes were unique (i.e., not shorted to each other). The data were filtered using a Butterworth high pass filter to remove motion artifacts. Artifacts were detected and corresponding timestamps obtained. Artifacts were removed from the raw data based on artifact timestamp locations. The data was then stitched. The data was then filtered between 300-5000 Hz to remove out-of-band interferers and help find ENG signals.

Immunohistology: Following electrophysiologic studies, the implant with the distal 2 cm of nerve was extracted en-bloc and placed in 10% buffered formalin as per the immunohistochemistry protocol (FIG. 29A). After 48 hours, the external silicone tube was removed without damaging the contents (FIG. 29B). Photographs of gross morphology were taken under 2.5× and 4× magnification. The tissue was then separated from the implant using microsurgical instruments under a dissecting microscope. The platinum coils were extracted from the tissue without causing breakage of the tissue to facilitate histologic sectioning. 7 μm sections were obtained using a standard microtome. The sections were subjected to H&E stain. Immunohistological labelling was performed with Neurofilament antibody (ABCAM , USA) for labeling axons, S-100 antibodies (ABCAM, USA) for Schwann cells, and CD45 antibodies (ABCAM, USA) for neutrophils. Standard protocols as recommended by the manufacturer were used. A 3 mm normal nerve segment was harvested for control.

Results

Implant Design, Fabrication and Testing

Our aim was to achieve a fine organization of regenerating axons within a three-dimensional textured guidance structure capable of providing a physical support and axonal guidance. The design of the guidance structure was based on our understanding of axonal guidance on biocompatible material surfaces as well as neurosurgical expertise. We constructed a device in accordance with the device 10 of FIGS. 1-8. The channels 19 had a textured surface to provide guidance support and a diameter of 1.34 mm-2 mm to provide enough transparency, i.e., free space for axons and supportive tissue to grow. The conductive elements 34, coiled stripped platinum/iridium (Pt—Ir) electrodes, were incorporated within two of the channels 19. The third channel was left empty to study tissue histo-morphology in the event that the tissue in the other two channels was damaged during extraction from the coil electrode. Insulated wires were used to connect the electrodes to an external custom-made connector. All connections were hermetically sealed.

Prior to implantation, the impedance of the construct was monitored during 15 week-long soak tests at 67°, as described above.

The experimental protocol was approved by the Institutional Animal Care and Use Committee (IACUC). Prior to the implantation, the construct was enclosed in a silicone cylinder and sterilized with ethylene oxide. We placed the macaque under general anesthesia. Following full sterile surgical preparation, we exposed the ulnar nerve in the arm and divided the nerve 5 cm proximal to the elbow. We then inserted the spire 16 of the device 10 into the inter-fascicular space (FIGS. 24 and 25). The column of the implant with the electrodes remained external to the nerve (FIG. 25). The implant was buried within the intermuscular plane. The connector was positioned in the subcutaneous tissue for subsequent access and recording after the nerve regeneration had taken place. An incubation period of four months was allowed after implantation. During this period, the macaque was allowed free movement, diet and enrichment as per our institutional protocol. Explantation and electrophysiology were carried out upon completion of four month duration.

Electrophysiologic conduction across the interface (FIG. 26)

Following general anesthesia, we performed a contralateral temporal craniotomy to expose the motor cortex. Following trial stimulations, we located the cortical region that elicited maximal intrinsic muscular contraction in the hand. We surgically exposed the subcutaneously placed connector in the upper limb and connected it via a custom-made electrical adaptor to an Intan (Intan Technologies, LLC) recording system, without disturbing the interface.

Cortical stimulation and recording: In each trial, 10 stimulation sets with the same stimulation amplitude were executed. Each set lasted for 500 ms with biphasic current of 200 μs pulse width, frequency of 300 Hz, train duration of 20 ms, and train frequency of 24 Hz. Nine trials were performed with different stimulation amplitudes, ranging from 100 μA to 260 μA at 20 μA increments. Signals were recorded for both the channels (FIGS. 26A, 26B). Signals were acquired from the interfacing electrodes using the Intan amplifier system. Complete series at various amplitudes can be seen in FIG. 33. The raw signals were filtered with Matlab (The MathWorks, Inc.) with 30-5000 Hz Butterworth bandpass filter of order three to remove motion artifacts and out-of-band interference. Then, electrical stimulation artifacts were identified by using a threshold method. After excluding the artifacts, CAP was identified for each stimulation protocol. The observed signals were within 2-5 ms time interval. SNR of the CAP evoked by different stimulation amplitudes was computed as shown in FIG. 26, which showed a linearly proportional relationship between SNR and stimulation amplitude. To analyze the recruitment response to different stimulation amplitudes, a signal threshold of 10 μV was set to select the stimulation trains that successfully evoked strong signals. FIG. 26 depicts the average peak voltage of CAP and the triggering probability of different stimulation amplitudes. Starting from 180 μA current, consistent CAP peak voltage with the mean of 14.38 μV and 13.76 μV could be observed in channel 1 and channel 2 respectively (p<0.05). Besides, triggering probability increased in scale of the stimulation amplitudes (FIGS. 26A-26D), which showed that more axons were successfully recruited when higher stimulation amplitude was used. Impedance measurement carried out indicated that both electrodes were unique (i.e., not shorted to each other).Filtered signals single stimulus series to show the stimulus train and the intervening action potentials in two channels can be seen in FIG. 26E.

Morphology: We removed the implant with a segment of the nerve en-bloc and placed in 10% buffered formalin for 48 hours for fixation (FIG. 27A). The external silicone cylinder was removed to make morphologic observations. New tissue growth from the nerve was seen extending from the normal nerve (n) and encapsulating the body of the implant 10 (FIGS. 27B, 27B1). This tissue was observed growing within the channels as well as encapsulating the intervening columns. The coil electrodes within the channels were internalized within this new tissue (FIG. 27B2). The tissue was firmly adherent to the implant. On separation from the implant (FIG. 27C), the new tissue growth (fxg) appeared as a sheet forming a hollow cylinder around the implant.

Histomorphology: We fixed the specimen in buffered formalin. Following fixation, we separated the specimen from the implant 10 (FIG. 27C). We extracted the coil micro-electrodes under magnification without damaging the tissue to facilitate histological sectioning. For histological analysis, we divided the tissue into four zones (FIG. 27D). The normal nerve (n), the transitional zone (t), leading to fibro-axonal growth (fxg) which was divided into proximal (fxp) and distal tip (fxd). Two specimens were sectioned transversely and one specimen was sectioned longitudinally. 7 μm thick histological sections were taken from the normal nerve, the transition zone and the new growth around the body of the implant. For the controls one specimen was sectioned transversely and one was sectioned longitudinally 7 μm thick sections were obtained.

Haematoxylin & Eosin (H&E) stain demonstrated a clear transition from normal nerve (FIG. 28C1) via a transitional zone (FIG. 28B1) to the new fibro-axonal growth (FIG. 28A1). The fibro-axonal growth consistently followed the contour of the implant creating a clover like configuration. The new growth showed three distinct zones. The inner fibro collagenous layer, the axonal zone and the outer collagenous layer. Both fibro-collagenous layers showed a well-organized parallel arrangement of fibroblasts (FIG. 28A1)

Immunohistology:

Neurofilament antibody labeling; Neuro-filament (NF) antibodies was performed to label the axons. It revealed a unique morphology. Normal nerve (n) with fascicular arrangement of axons was seen proximal to the implant 10 (FIG. 28C2). The transitional zone (t) (FIG. 28B2) between the normal nerve and the beginning of fibro-axonal growth demonstrated a laminar pattern of axons surrounding three lacunae representing the tips of the columns between the channels. The proximal fibro-axonal growth (FIG. 28A2) as well as distal fibro-axonal growth (FIG. 29, A1&A2) showed highly organized laminar arrangement of axons, as opposed to the fascicular pattern in the normal nerve forming a well-defined layer (dark stain). This layer was sandwiched between layers of collagen fibers and fibroblasts (light stain). This layered sheet of tissue precisely followed the contour of the implant and completely filled up the channels where the electrodes were located.

We measured the thickness of the inner fibrous layer in five sections for two specimens processed for transverse sectioning: The thickness varied from 25 μm in the narrowest zone to 100 μm in the widest zone (mean 70.7±15 μm). The outer fibrous layer was consistently thicker and measured in the range of 110-250 μm (mean thickness 196.5±43 μm). The axonal layer, measured in five sequential sections in three specimens, ranged from 50-450 μm (mean 344±45 μm). Axonal density was calculated by selecting 20 random 100×100 μm fields of NF staining on sections in the transversely sectioned specimens. The axons appear in clusters of 40-100 axons. The density ranged from 120 to 400 axon per 104 μm2, i.e., three to ten clusters per field. The longitudinal section also demonstrated the fibro-axonal growth and a layered arrangement of axons extending into the implant channels (FIG. 29B1,B2, FIG. 30A1,A2).

S100 antibody stain (FIG. 30A2): S100 antibodies were used to label Schwann cells. Labelling demonstrated complete topological co-location of Schwann cells with the NF positive axons indicating that the axons were myelinated.

CD45 stain (FIG. 34): demonstrated near absence of inflammatory cells (neutrophils) in the composite tissue. We observed sporadic 1-2 cells per high power field. There was no evidence of micro-fragmentation or phagocytosis of implant material within the tissue indicating absence of ongoing inflammatory response.

Neurofilament staining in control specimen (without the implant 10): FIG. 2C1 and C2 show a typical end-neuroma formation at the cut end of the nerve. Its morphology is representative of an un-manipulated endpoint of nerve transection in absence of an implant. Immunohistology with NF labelling demonstrated random orientation of axon clusters (FIG. 29C1) without the characteristic laminated configuration of axons seen in the specimens with the implant. A comparison is seen in the transverse sections in FIGS. 29A2 and 29C1, and longitudinal sections in FIGS. 30A1 and 30B1.

Discussion

Stable long-term neural interfaces are the key to the development of neuro-prostheses. Conventional methods such as extraneural FINE electrodes or penetrative intraneural arrays (Utah microarray, LIFE, TIME) are suitable for recording or stimulation for limited durations. This is largely due to the inherent trauma and subsequent fibrosis induced by the electrode itself. In contrast, a biohybrid system refers to a construct that harbors biologic and a-biologic components in a stable relationship over a long period of time and can be translated into a permanent or near permanent implant.

Fibrosis has been the unresolved challenge in previous attempts to create neural interfaces. For example, in previous implants, a flat electrode is provided in the wall of a microchannel device. However, fibrotic growth on the electrode surface creates insulation between the axons and the electrode surface. Various materials have been used to coat the electrodes to enhance axonal guidance, but this does not address the issue of fibrotic sequestration.

Distinct from the existing approaches, the presently disclosed implant accommodates fibrosis as a part of the interface, and to maintain electrophysiological contact with the axons within the paradigm of fibrosis.

The design of the presently disclosed implant is based on the following aspects of peripheral nerve biology:
When a peripheral nerve is injured, a brief phase of Wallerian degeneration is followed by axonal regeneration. The regenerating axons are guided by Schwann cells in the distal segment of the nerve to re-form parallel fascicles. In case of amputations, where the distal end is unavailable to provide guidance, the unguided axons form an ‘end-neuroma’, which is a disorganised mass of axons trapped within mature fibrous tissue (FIG. 30C,C1,C2).
The neuroma is thus a product of two parallel processes taking place at the end of an injured nerve. First, the unguided axonal regeneration and second, the local organization of fibrous tissue for tissue healing. However, it is interesting to note that although these axons are randomly organized and trapped within a mass of fibrous tissue, they continue to retain their electrophysiological activity. The term ‘fibro-axonal tissue’ refers to this composite of axons and fibrous tissues.
End neuromas from peripheral nerves are consistently encountered at the site of limb amputations, where the cut ends of peripheral nerves attempt to regenerate without the availability of a distal end. Accessing axons within these neuromas is a practical means for interfacing with the prosthesis.

The possibility of a neural interface with fibro-axonal composite tissue was proposed by Lahiri et al in the rat sciatic nerve. However, their model was based on spontaneous self-organization of tissues directly on the electrodes.

Our aim was to guide this fibro-axonal growth on a biocompatible surface and use design strategies to obtain enhanced contact between the electrodes and the axons within this fibro-axonal composite and demonstrate this concept in the macaque model.

Accordingly, an implant 10 was designed with a pedestal 12 and an elongated spire 16 (FIGS. 1-8). The spire 16 may be 20 mm long with a blunt tip, for non-traumatic insertion into the inter-fascicular space of the nerve (FIG. 31A). The body may have longitudinal grooves 30 with width (W) and depth (D) gradually decreasing from W=0.72 mm and D=0.335 mm to W=0.51 mm to D=0.2 mm at the column 22. The purpose of the grooves 30 is to allow ingrowth of the fibro-axonal tissue originating from the cut end of the nerve and to maintain axial alignment of the column to the nerve.

The column 22 may be 10 mm long and 4 mm in diameter, which matches the diameter of the ulnar nerve in the macaque and allows axonal growth onto the surface. The column 22 may form three channels 19 having 1.34 mm diameter (FIG. 4). The column walls have a textured surface with longitudinal grooves 0.51 mm wide, 0.2 mm deep and angle of 71.91° between the edges (FIG. 4). This creates a linear orientation and their growth toward the contact with the electrodes. The implant (FIG. 1) was made by 3-D printing VeroClear™ (RGD810, Stratasys Limited). Our decision to use this material, and the channel dimensions, were based on the in vitro data discussed above.

The implant 10 provided a substrate for axonal growth and reconfigured the tissue in several different aspects. The fibro-axonal tissue generated at the cut end of the nerve which was destined to form an end neuroma was re-configured into a sheet of tissue around the implant with ingrowth of the tissue into the channels (FIGS. 28, 29) creating a hollow cylinder of solid tissue contoured to the shape of the implant. The normal group-fascicular arrangement of axons in the nerve was transformed into a thin laminar distribution within this sheet of tissue. This was achieved in three ways: (i) by providing nerves with a rigid substrate to adhere and grow on; (ii) by parallel texturization of the substrate to enforce directional outgrowth; (iii) confinement of the available space to limit blind sprouting of regenerating axons. Texturization of the substrate into parallel microgrooves was shown to induce alignment of the fibro neuronal tissue in vitro (see results above). Accordingly, in the longitudinal section, the tissue growth into the channels showed linear and parallel arrangement of axons (FIGS. 30A1,A2). In the control group the cut end of the nerve without the implant demonstrated a typical neuroma formation (FIGS. 29C,C1, 30B,B1) with random arrangement of axons due to unguided growth underscoring the role of the implant.

It is important to note that for the above phenomena to occur, the implant material should be rigid and biocompatible but not biodegradable. It should be able to provide a solid substrate for fibro-axonal growth and maintain the spatial locations of the electrodes.

One important aspect of the implant design for neural interface applications is the use of coil electrodes that are embedded within the aforementioned channels. It was found that this resulted in internalisation of coils within the fibro-axonal growth into these channels. Although the axons were embedded between layers of fibrous tissue, the circumference of the coil within the tissue intersected with the axonal layer at multiple sites (FIG. 27B2, FIGS. 31A-D), creating bio-electrical contact points. In other words, the electrodes were able to make contact with axons even though the axons were located within layers of fibrous tissue.

This design was more effective compared to previously reported sieve or micro-channel designs where the electrodes were designed as flat surfaces. These flat surfaces were more likely to lose contact with the axons once fibrosis set in. This difference is illustrated in FIG. 31E. The electrodes were made from 2.4 cm long, bare Pt—Ir wire (diameter 0.05 mm) shaped into a 1 cm long coil (diameter approx. 0.85 mm). When located in the channel, its distance from the walls was 0 mm to 0.3 mm. The circumference of the coil is large enough to be able intersect the entire thickness of the tissue within the channel.

Another important observation in our study was the absence of inflammatory cells (neutrophils) in H&E (FIG. 28) stain as well as CD45 (FIG. 34) stain and the presence of well-organized layers of fibro-collagenous tissue. This indicated that there was no ongoing inflammatory reaction to the material and the collagen encapsulation was likely to remain stable. We also found strong co-localization of Schwann cells with the axons which demonstrated that that the nerve fibers were myelinated, having achieved biological maturity.

Well organized collagen and mature axons strongly endorse the biological stability of this construct. The absence of material breakdown, or phagocytosis, and absence of inflammatory cells indicates the biocompatibility and in-vivo stability of the material.

Functionality of this construct was conclusively demonstrated by detection of cortical signals across the neural interface. Starting from 180 μA amplitude stimulation, consistent CAP peak voltage with the mean of 14.38 μV and 13.76 μV could be observed in channel 1 and channel 2 respectively (p<0.05). These amplitudes represent pure motor action potentials detected from the nerve. Normally motor conduction is represented by CMAPs which measures voltages from the muscles in millivolts (mV), and sensory action potentials (SNAPs) are measured directly from nerves in microvolts (μV). However, in our studies we measured motor action potentials (MAPs) directly from the axonal interface as the nerve was completely separated from the recipient muscles. As of now there is no comparable data available for macaques. Normal values for SNAP in rhesus monkey are 14.6±9.4 μV. Our values of MAPs were in a similar range.

It is also important to note that during the 4 month period following the implant, the macaques continued with their normal activities. They did not show any signs of pain or discomfort at the site of the interface. There was no incidence of impaired wound healing or extrusion of the implant.

Our unique approach for a biohybrid interface can be summarized as follows:
We devised a novel contoured biocompatible implant to obtain tissue encapsulation and effectively transformed the structure of a neuroma into a sheet like configuration of fibro-axonal tissue. This configuration made the axons more accessible and organized compared to a solid mass of tissue seen in the control group (FIGS. 26, 27, 28).
The channels allow ingrowth of fibro-axonal tissue. The presence of coil electrodes within these channels allowed predictable encasement of the electrodes within the tissue (FIGS. 27, 28)
The configuration and the circumference of the coil allowed the coil to intersect the axonal layer at multiple points (FIGS. 26, 31). This concept proved more effective than placement of conductive surface in the walls of channels and was able to maintain contact with the axons that were encased within the fibrous tissue.
The fibrous encapsulation created a strong anchor between the nerve and the implant and played a structural role in the interface.
Our model overcame the problem of reactive fibrosis by using implant design to enable the growth of contoured fibro-axonal composite tissue, in effect making reactive fibrosis a part of the interface, while still maintaining electrophysiologic contact with the axons, thus creating a stable interface.

The experiments reported here, carried out in 3 macaques, provided a proof of concept of this novel approach to construct a biohybrid system to serve as a long term implantable neural interface.

As discussed above, a neural interface based on axon guidance using the device (such as guide element 10) according to certain embodiments can be connected with a wireless implant package to transmit the signal to an external decoding set up. From there, the signal can be translated into desired movement of, for example, a neuroprosthesis or a robotic assistant.

Many modifications will be apparent to those skilled in the art without departing from the scope of the present invention.

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1-20. (canceled)

21. An implantable guide element, comprising:
a main body formed from a biocompatible material; and
one or more grooved or ridged surface structures on and/or within the main body, each grooved or ridged surface structure comprising one or more grooves or ridges for directionally guided growth of, and encapsulation by, fibro-axonal tissue.

22. The implantable guide element according to claim 21, wherein at least one of the one or more grooved or ridged surface structures forms a channel along or within the main body.

23. The implantable guide element according to claim 22, comprising an electrode disposed within the channel and spaced from a wall of the channel along at least part of its length.

24. The implantable guide element according to claim 23, wherein the electrode has a helical portion.

25. The implantable guide element according to claim 21, wherein at least one of the one or more grooved or ridged surface structures has a coating comprising one or more of: charge changing molecules, adhesion molecules, growth factors, and supportive cells.

26. The implantable guide element according to claim 25, wherein the coating has a concentration gradient.

27. The implantable guide element according to claim 25, comprising two or more grooves or ridges having different respective coatings suitable for promoting adhesion and/or growth of different respective cell types.

28. The implantable guide element according to claim 21, wherein the main body is an elongate structure having an axis, and the at least one grooved or ridged surface structure is aligned generally along the axis.

29. The implantable guide element according to claim 21, wherein the main body has a tapered end for insertion into a nerve.

30. The implantable guide element according to claim 23, wherein the main body has a rigid base portion.

31. The implantable guide element according to claim 30, wherein the rigid base portion houses a connector of the electrode.

32. The implantable guide element according to claim 30, wherein the rigid base portion houses one or more of an innervation target, one or more molecular growth factors, a source of a magnetic or electromagnetic field, and one or more guidance molecules.

33. The implantable guide element according to claim 21, wherein the biocompatible material is VeroClear.

34. A method of fabricating a guide element for implantation into a subject, comprising:
obtaining dimensional measurements of a nerve of the subject; and
forming, in accordance with the dimensional measurements by an additive manufacturing method, using a biocompatible material:
a main body; and
one or more grooved or ridged surface structures on and/or within the main body, each grooved or ridged surface structure comprising one or more grooves or ridges for directionally guided growth of, and encapsulation by, fibro-axonal tissue.

35. The method according to claim 34, wherein at least one of the one or more grooved or ridged surface structures forms a channel within the main body.

36. The method according to claim 35, comprising providing an electrode within the channel and spaced from a wall of the channel along at least part of its length.

37. The method according to claim 36, wherein the electrode has a helical portion.

38. The method according to claim 34, comprising applying a coating to one or more of the one or more grooved or ridged surface structures, the coating comprising one or more of: charge changing molecules, adhesion molecules, growth factors, and supportive cells.

39. The method according to claim 38, comprising applying the coating with a concentration gradient.

40. The method according to claim 38, comprising applying different respective coatings suitable for promoting adhesion and/or growth of different respective cell types to two or more of the grooved or ridged surface structures.
